(19) United States
(12) Patent Application Publication
(10) Pub. No.: US 2022/0403345 A1
(43) Pub. Date: Dec. 22, 2022

(54) A MAMMALIAN-AVIAN CHIMERIC MODEL SYSTEM

(71) Applicant: INNOVO MIMETICS LIMITED, Jerusalem (IL)

(72) Inventors: Robert Edwin GOLDSTEIN, Kibutz Tel Yosef (IL); Avner YEFFET, Jerusalem (IL); Julia RIFMAN, Modiin (IL); Leah APELBAUM, Jerusalem (IL); Vered BEN-HUR, Jerusalem (IL); Tamara LANDAU, Jerusalem (IL); Inbal DORON, Jerusalem (IL)

(21) Appl. No.: 17/767,176

(22) PCT Filed: Oct. 7, 2019
(86) PCT No.: PCT/IL2019/051102

§ 371 Date: Apr. 7, 2022

(51) Int. Cl. C12N 5/071

(52) U.S. Cl. CPC C12N 5/0697; C12N 2501/20; C12N 2501/2302; C12N 2502/1164; C12N 2502/28; C12N 2533/90

(57) ABSTRACT

The present invention is directed to a mammalian-avian chimeric model system comprising a fertilized avian egg comprising a chorioallantoic membrane (CAM); and multiple types of mammalian cells dispersed in a hydrogel. Further provided is a method for preparing the system and a method of using the same.

### CROSS REFERENCE TO RELATED APPLICATIONS

This application claims the benefit of priority of U.S. Provisional Patent Application No. 62/742,488 titled “MAMMALIAN-AVIAN CHIMERIC MODEL SYSTEM”, filed Oct. 8, 2018, the contents of which is incorporated herein by reference in its entirety.

### FIELD OF INVENTION

The present invention is in the field of chick embryo chorioallantoic membrane model systems.

### BACKGROUND

Many in vivo systems, particularly model systems for studying human tissues and organs, require transplanting an examined graft into an immune-compromised animal, in order to avoid graft rejection. Such animals, for example, severe combined immune deficient (SCID) mice and NOD scid gamma (NSG) mice, are expensive, sickly and difficult to maintain. To study the activity of human immune cells in these mice requires additional genetic modifications to the host, as well as treatment of the mice with a cocktail of human cytokines to support immune cell function. One feature of animal xenograft tumors is the formation of a tumor micro-environment by the cancer cells and surrounding stroma cells.

The avian egg provides a low-cost and readily available alternative to in vivo testing in sentient animals. The avian egg has been used in growth of viruses for vaccine generation, angiogenesis assays, teratogenicity testing, tumor cells and the like.

Currently, there is no available method for studying the interactions between multiple types of foreign cells. e.g., immune cells and cancer cells, and the influence of drugs on these interactions, in the avian egg, or more generally, for controlling or applying in a predictable manner a system of introducing multiple cell types into direct contact with each other within the living egg.

### SUMMARY

The present invention, in some embodiments thereof, is directed to a mammalian-avian chimeric model system comprising a fertilized avian egg comprising a chorioallantoic membrane (CAM) engrafted with multiple types of mammalian cells, wherein each of the mammalian cells is dispersed in a hydrogel. The present invention is further directed to a method of preparing the mammalian-avian chimeric model system, and a method of use thereof, such as for screening for therapeutic compounds.

According to a first aspect, there is provided a mammalian-avian chimeric model system comprising: (i) a fertilized avian egg comprising a chorioallantoic membrane (CAM); (ii) a first type of a mammalian cell; and (iii) a second type of a mammalian cell, wherein the first type of a mammalian cell and the second type of a mammalian cell are dispersed in separate hydrogels, and wherein the separate hydrogels are at a distance of not more than 5 mm from one another on the CAM.

According to another aspect, there is provided a method for preparing a mammalian-avian chimeric model system, comprising: (i) providing a fertilized avian egg comprising a CAM; (ii) grafting a first type of a mammalian cell dispersed in a hydrogel to the CAM; and (iii) grafting a second type of a mammalian cell dispersed in a hydrogel to the CAM, thereby preparing a mammalian-avian chimeric model system.

According to another aspect, there is provided a method for determining an interaction between a first type of a mammalian cell and a second type of a mammalian cell, comprising: (i) providing the herein disclosed mammalian-avian chimeric model system; and (ii) determining at least one of: (a) cellular phenotype in the first type of a mammalian cell; and (b) an effector activity in the second type of a mammalian cell, wherein a change of the phenotype in the first type of a mammalian cell and/or a change of the effector activity in the second type of a mammalian cell compared to control, is indicative of an interaction between a first type of a mammalian cell and a second type of a mammalian cell.

According to another aspect, there is provided a method of screening for a compound suitable for treating a disease associated with cell proliferation, comprising the steps: (i) providing the herein disclosed mammalian-avian chimeric model system; and (ii) contacting the system with a compound and determining a phenotype in the first type of a mammalian cell, wherein a change of the phenotype in the first type of a mammalian cell in the presence of the compound compared to the phenotype of the first type of a mammalian cell in the absence of the compound is indicative of the suitability of the compound in treating a disease associated with cell proliferation.

In some embodiments, anyone of the separate hydrogels comprises 50,000 to 1,000,000 cells.

In some embodiments, the first type of a mammalian cell is a proliferating cell or a differentiating cell.

In some embodiments, the proliferating cell is an abnormally proliferating cell or a cancerous cell.

In some embodiments, the second type of a mammalian cell is an immune cell, an endothelial cell, or any progenitor cells thereof.

In some embodiments, the immune cell is selected from the group consisting of: an infiltrating cell, a cytokine-mediated remote killing cell, and a cell-cycle arresting cell.

In some embodiments, the immune cell is a lymphocyte or a myeloid cell.

In some embodiments, the system further comprises a therapeutic agent selected from the group consisting of: a chemical, a molecule, a polypeptide, a cell, and a virus.

In some embodiments, the system further comprises one or more stimulatory agents, wherein the one or more stimulatory agents increases one or more cellular activities selected from the group consisting of: proliferation, growth, survival, motility, angiogenesis, neo-vascularization, and cytotoxicity.

In some embodiments, at least a portion of the cancerous cells are in a form of a tumor or a cystoid.

In some embodiments, each of the hydrogels is provided to the CAM at a distance of not more than 5 mm from one another.

In some embodiments, the hydrogels are grafted to the CAM on embryonic day 6 (E6) to embryonic day 17 (E17).

In some embodiments, the hydrogels are provided simultaneously.

In some embodiments, the hydrogels are provided sequentially.

In some embodiments, sequentially comprises grafting the first type of a mammalian cell followed by grafting of the second type of a mammalian cell.

In some embodiments, sequentially grafting comprises 1 to 96 hours between each grafting.

In some embodiments, the method further comprises a step of determining the engraftment of the first type of a mammalian cell to the CAM.

In some embodiments, the method further comprises providing one or more stimulatory agents to one or more of the first type of a mammalian cell and the second type of a mammalian cell.

In some embodiments, the one or more stimulatory agents increases one or more cellular activities selected from the group consisting of: proliferation, growth, survival, motility, angiogenesis, neo-vascularization, and cytotoxicity.

In some embodiments, the stimulatory agent is provided to the first type of a mammalian cell, the second type of a mammalian cell, or both, before engraftment to the CAM, after engraftment to the CAM, or both.

In some embodiments, the providing is by: contacting one or more of the first type of a mammalian cell and the second type of a mammalian cell, an intravenous injection into the CAM blood vessels, or a combination thereof.

In some embodiments, the method further comprises a step of determining the effector activity of the second type of a mammalian cell.

In some embodiments, the effector activity is selected from the group consisting of: infiltration, cytokine secretion, cytokine-mediated remote killing, and cell-cycle arresting.

In some embodiments, the method further comprises a step of contacting the mammalian-avian chimeric model system with one or more stimulatory agent, wherein the one or more stimulatory agents increases one or more activities selected from the group consisting of: proliferation, growth, survival, motility, angiogenesis, neo-vascularization, and cytotoxicity.

In some embodiments, the cellular phenotype of the first type of a mammalian cell is selected from the group consisting of proliferation rate, cell death rate, differentiation, tumorigenesis, and metabolic rate.

In some embodiments, the effector activity of the second type of a mammalian cell is selected from the group consisting of: infiltration, cytokine secretion, cytokine-mediated remote killing, cell-cycle arresting, angiogenesis, and neo-vascularization.

In some embodiments, the second type of a mammalian cell is an immune cell, an endothelial cell, or progenitor cells thereof.

In some embodiments, the disease associated with cell proliferation is cancer or psoriasis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C are micrographs showing bright field images of a Matrigel® pellet formed comprising 3×10⁵ cells of PEA2 ovarian cancer cell line. (1A) is an image of a liquid Matrigel® placed on Parafilm™ at 37° C. for 30 minutes and allowed to solidify before being placed on the CAM of an embryonic day 9 egg (E9; pellet circumference=3-5 mm). (1B) is an image of a tumor excised from an E16 egg following engraftment with a MATRIGEL® pellet, as shown in (F1A). The egg was treated with 400 μg Carboplatin by intravenous injection at embryonic day E13 and had shrunk in size compared to the original MATRIGEL® pellet. (1C) is images of a low-passage ovarian cancer cell line derived from ascites liquid drained from a patient which was engrafted, and the resulting tumor was excised, fixed embedded, sectioned (7 μm) and stained with either (left) hematoxylin and eosin or (right) for expression of CD44 by immunohistochemistry (tumor diameter is ˜0.4 cm).

FIGS. 2A-2F are schematic illustrations of non-limiting examples of the disclosed invention describing cell-culturing and co-engraftments. (2A) is a schematic diagram of cancer cells and immune cells cultured separately that are mixed and solidified in a single matrix pellet and engrafted onto the chorioallantoic membrane (CAM), also termed a “Winn” assay. (2B) is a schematic diagram of cancer cells and immune cells cultured separately that are solidified in 2 separate matrix pellets, and are then engrafted adjacently to one another, but without direct contact, onto the CAM. (2C) is a schematic diagram of cancer cells and immune cells cultured separately. Initially the cancer cells are solidified in a matrix pellet and are engrafted onto the CAM. Once a tumor has been formed, the immune cells are solidified in a matrix pellet and are engrafted either directly on top of the newly formed tumor, or onto the CAM while touching the tumor. (2D) is schematic diagrams of the apical views (1 and 2) and cross-section views (3 and 4) of the two matrix pellets described in (2B), placed on the CAM of a fertilized E9 egg (with a shell cut away). The pellets start off at a short distance from one other on the CAM (1 and 3), and then during the next 1-3 days of incubation end up drawing close to each other and form a tumor (2 and 4). (2E) is a schematic diagram of cancer cell and immune cell distribution within the two matrix pellets that have formed a single tumor (D2 and D4). Simulations of cells distribution in which (1) immune cells do not invade the cancer cell matrix pellet; (2) immune cells invade the cancer cell matrix pellet but do not kill the cancer cells; and (3) immune cells invade the cancer cell matrix pellet and kill the cancer cells, are presented. (2F) is a schematic diagram of two cell cultures in two matrix pellets on a single CAM where each matrix pellet has been formed containing a different exogenous agent.

FIGS. 3A-3O are micrographs depicting a “Winn” assay in an egg tumor xenograft model. Tumor infiltrating lymphocytes (TILs) were generated from patient melanoma tumor tissue. TILs 463 are HLA-A2 positive (3B, 3D, 3G, 3I, 3L, and 3N); control TILs (425) are non-melanoma specific (3E, 3J and 3O). TILs and melanoma cells Mel-624 (M-624; HLA-2A positive cell line) or Mel-888 (M-888; non-HLA-2A cell line) were mixed separately and then formed into matrix pellets either separately or in a mix with TILs (at 1:1 ratio). (3A-3E) is 5 micrographs comprising sets of 6 tumors derived from (3A) M-624; (3B) M-624:TILs 463; (3C) M-888; (3D) M-888:TILs 463; and (3E) M-624:TILs 425, all after 5 days of incubation following engraftment. Tumors diameter ˜5 mm; scale bar=1 cm. (3F-3O) is micrographs of the tumor compositions of (3A-3E) harvested 48 hours after engraftment. Tumor sections were immune-stained for a proliferation marker (Ki67; 3F-3J) and for an apoptosis marker (cleaved Caspase-3; 3K-3O). A visual score for staining was devised as follows: (+++) staining in a large proportion of cells; (+) staining in a low proportion of cells; and (−) very little staining. Results were as follows: 3F (+++); 3G (—); 3H (+++); 3I (+++); 3J (+++); 3K (—); 3L (+++); 3M (−); 3N (+); and 3O (−).

FIGS. 4A-4D are micrographs of histological tissue sections of immune-stained T-cells in in ovo tumors. (4A) Mel-888+TILs 463 at 48 h incubation post-engraftment; and (4B) Mel-624+TILs 463, at 120 h incubation post-engraftment (in B), were co-stained by for the human cell marker LAMP-1 and for the CD8 epitope restricted to human cytotoxic T-cells. (4C-4D) are higher magnification images of the black square region shown in (4A-4B), respectively.

FIGS. 5A-5B are images of egg CAM taken by camera through an opening in the apical end of the eggshell. (5A) two matrix pellets (Cultrex® (BME3)) engrafted side by side on the CAM on E9, containing cancer cells (Mel-624) and immune cells (patient Natural killer cells (NKs)). (5B) 48 hours later the same egg was documented, showing a newly formed tumor on the CAM that is half gray (left arrow) and half white (right arrow) in appearance, indicating the two matrix pellets (5A) fused into a single tumor.

FIGS. 6A-6J are micrographs of histological tissue sections of the experimental groups shown in FIG. 5, harvested 48 hours after engraftment, and a graph. Serial tissue sections were immune-stained for a melanoma marker (MelanA; 6A, 6D and 6G), for the NK cell marker (CD56; 6B, 6E and 6H) and for an apoptosis marker (cleaved Caspase 3; 6C, 6F and 6I). (6A-6C) are control tumor containing only a Mel-624 matrix pellet. (6D-6F) are images of serial sections from a tumor (T2) containing a Mel-624 matrix pellet and a NK cell pellet that have fused, and where the NK cells (on the right side of each image) did not invade the part of the tumor containing the cancer cells (left side of the image; scale bar=100 μm). (6G-6I) are images of a tumor (T5) containing a Mel-624 matrix pellet and a NK cell pellet that have fused, and where the NK cells have invaded the part of the tumor containing the cancer cells. (6J) is a graph showing a clear inverse relationship between the number of CD56 positive cells per image and the number of melanoma cells per field (5 independent tumors (tumors T2 and T5 are indicated on the graph).

FIGS. 7A-7D are micrographs of histological sections of fused tumors comprising two separate sets of matrix pellets prepared from cell suspensions of melanoma cells and TILs. Tumor infiltrating lymphocytes (TILs) were generated from a patient melanoma tumor tissue (termed “first patient TILs”). Pellets were engrafted onto the CAM at a distance of <1 mm. Serial tissue sections of two different tumors (first tumor; 7A-7B and second tumor; 7C-7D) were immunostained for a lymphocyte marker (CD45; 7A and 7C), melanoma marker (MelanA; 7B and 7D). (7A and 7C) TILs were not shown to infiltrate the compartment of the tumor containing the melanoma cells, or vice versa (7B and 7D). The partition between the two compartments wherein each type of cell was suspended is demarcated with a black dashed line.

FIGS. 8A-8D are micrographs of histological sections of two fused tumors comprising two separate sets of matrix pellets prepared from cell suspensions of melanoma cells and TILs from a second patient, respectively. Pellets were engrafted onto the CAM at a distance of <1 mm from one another, and subsequently a tumor formed containing a fusion of the two pellets. Serial tissue sections of a tumor were immunostained for a lymphocyte marker (CD45; 8A and 8C), melanoma marker (MelanA; 8B and 8D). (8A and 8C) TILs were shown to infiltrate the compartment of the tumor containing the melanoma cells (on the right side of the image—a dashed black line demarcates the border between the TILs and the melanoma cell compartments).

FIGS. 9A-9D are micrographs of histological tissue sections of two combinations of cell pairs comprising Jurkat cells with either (9A) Mel-624 or (9B) B16. The Jurkat cells mixed with the second cell type without breaking off as invading individual cells, inversely, neither B16 nor Mel-624 showed significant infiltration of single cells into the Jurkat compartment. (9C-9D) are higher magnifications of (9A-9B), respectively. Jurkat cells were identified as being CD3 positive. Scale bar=100 μm (9A-9B) and 50 μm (9C-9D).

FIGS. 10A-10L are micrographs of histological sections of fused tumors comprising two separate sets of matrix pellets prepared from cell suspensions of melanoma cells (Mel 624) and either PBMC or TILs from the third patient. Pellets were engrafted onto the CAM at a distance of <1 mm from one another, and subsequently a tumor formed containing a fusion of the two pellets. Matrices were either diluted with PBS, serum, or not diluted. (10A-10C) PBMC: Mel 624, undiluted matrigel; (10D-10F) PBMC: Mel 624, 50% serum and 50% Matrigel; (10G-10I) TILs from the third patient: Mel 624, 50% serum and 50% matrigel; and (10J-10L) TILs from the third patient: Mel 624, 50% PBS and 50% matrigel. Sections were immunostained (with DAB) for a lymphocyte marker, CD45, and counterstained with Hematoxylin to identify nuclei (10A, 10D, 10G, and 10J). ImageJ was used to create a mask for Melanoma cells that were identified by morphology and nuclear size using Hematoxylin stain (10B, 10E, 10H, and 10K). Similarly, ImageJ was used to create a mask for cells stained with DAB indicating CD45 positive immune cells (10C, 10F, 10I, and 10L). A dashed line demarcates the border between the PBMCs/TILs and the melanoma cell compartments.

FIGS. 11A-11T are micrographs of histological sections of fused tumors comprising two separate sets of matrix pellets prepared from cell suspensions of TILs from the second, third, of fourth patient, PBMCs, or CAR-T with either melanoma cells (Mel 624) or BT474 breast cancer cells in the case of CAR-T. Pellets were engrafted onto the CAM at a distance of <1 mm from one another, and subsequently a tumor formed containing a fusion of the two pellets. (11A-11D) TILs from the second patient: Mel 624; (11E-11H) TILs from the third patient: Mel 624; (11I-11L) TILs from the fourth patient: Mel 624; (11M-11P) PBMCs: Mel 624; and (11Q-11T) CAR-T: BT474. Sections were immunostained (with DAB) for a lymphocyte marker, CD45, and counterstained with Hematoxylin to identify nuclei (11A, 11E, 11I, 11M, and 11Q). ImageJ was used to create a mask for Melanoma cells or BT474 breast cancer cells that were identified by morphology and nuclear size using Hematoxylin stain (11B, 11F, 11J, 11N, and 11R). Similarly, ImageJ was used to create a mask for cells stained with DAB indicating CD45 positive immune cells (11C, 11G, 11K, 11O, and 11T). A solid/dashed line demarcates the border between the PBMC/TILs and the melanoma cell compartments. Staining of caspase-3 (DAB) was applied so as to identify cells undergoing apoptosis (11D, 11H, 11L, 11P, and 11T).

FIGS. 12A-12F are micrographs of histological sections of tumors. (12A-12B) are tumors comprising a single matrix pellet prepared from a mixture of two cell suspensions: human umbilical vein endothelial cells (HUVEC) and human ovarian cancer cells (A2780 cell line). (12C-12F) are micrographs of histological sections of fused tumors comprising two separate sets of matrix pellets prepared from cell suspensions of HUVEC and human ovarian cancer cells (A2780 cell line). Cells were immunostained for the markers CD31 (12A-12C; used to detect HUVEC), and CD155 (12D-12F; used to detect A2780 cells). HUVEC cells appear in two distinct morphological forms in the A2780 cancer cell tumor: one form is a cluster of cells, the other form is as an elongated structure possibly with a vacuous center, where they have been incorporated into blood vessels (12A). While, HUVEC engrafted in a separate matrix pellet showed distinct localization of each of the two morphological forms, whereby within Side A the HUVEC form dense clusters of cells, and wherein Side B (where they have infiltrated the A2780 compartment) they are incorporated wholly into chick blood vessels (12B-12C). Scale bar=100 μm. In (12B and 12E), Side A matrix pellet comprised Matrigel® and 10 ng bFGF and HUVEC cells, while Side B comprised Matrigel® and 30 ng bFGF and A2780 cells. In (12C and 12F), Side A matrix pellet comprised Matrigel® 10 ng bFGF and HUVEC cells, while Side B comprised Matrigel® and gelatin sponge (Spongostan®), 30 ng bFGF and A2780 cells. HUVEC cells were incorporated into blood vessels in the A2780 compartments more frequently when the A2780 cells were engrafted in Matrigel®, rather than in Matrigel® and Spongostan®.

FIG. 13 is a micrograph showing a tissue section from a HUVEC—A2780 fusion tumor which was stained with an anti-CD31 (DAB) and hematoxylin (all nuclei). A circular blood vessel was found to comprise mostly of DAB stained cells (outlined with a dashed line). Cells which did not express CD31 (indicated by star symbols, and presumably chick endothelial cells) were also shown to be incorporated in the blood vessel. Scale bar=20 μm.

### DETAILED DESCRIPTION

According to some embodiments, the present invention is directed to a chimeric system comprising a fertilized avian egg and multiple types of xenografted cells, wherein the xenografted cells are dispersed in hydrogels while grafted to the egg's CAM.

In some embodiments, xenografted cells according to the invention comprise any cell originating (e.g., derived or isolated or obtained) from any non-avian organism.

In some embodiments, the xenografted cell is selected from the group consisting of a mammalian cell, a non-avian animal cell, bacteria, yeast, fungus, and a plant cell.

In some embodiments, the present invention is directed to a method for preparing the chimeric system, comprising dispersing the xenografted cells in one or more hydrogels, and engrafting the hydrogel-dispersed cells to the avian egg's CAM.

The invention is based, in part, on the finding that xenografted cells, such as proliferating cells (e.g., cancer cells) and immune cells, which were individually engrafted in separate hydrogels to a fertilized avian egg's CAM, subsequently fused and formed a single tumor comprising both cell types, thereby serving as a suitable model that is comparable to other in vivo systems. Without being bound to any theory or mechanism of action, it is suggested that providing each cell type to the CAM, while dispersed in separate hydrogels, allows the initial formation of a separate microenvironment originating from each cell type, prior to the interaction of one cell type on another.

### Cells

In some embodiments, a xenografted cell of the invention is a mammalian cell.

As used herein, the term “mammalian” or “mammal” refers to any organism belonging to the Mammalia class.

Non-limiting examples of mammals include, but are not limited to, antelope, bear, beaver, bison, boar, camel, caribou, cattle, deer, elephant, elk, fox, giraffe, goat, hare, horse, ibex, kangaroo, lion, llama, moose, peccary, pig, rabbit, seal, sheep, squirrel, tiger, whale, yak, and zebra.

In some embodiments, the mammal is a human subject.

In some embodiments, the mammalian cell is isolated from a mammalian subject. In some embodiments, the mammalian cell is derived from a mammalian subject. In some embodiments, the mammalian cell is isolated or derived from a prenatal form of a mammal, such as an embryo or a fetus. In some embodiments, the mammalian cell is obtained from a mammalian organ culture. In some embodiments, the mammalian cell is obtained from a mammalian tissue culture. In some embodiments, the mammalian cell is obtained from a mammalian cell culture. In some embodiments, the mammalian cell is obtained from a mammalian cell line. Non-limiting examples of cell lines include, but are not limited to, primary cell line, immortalized cell line, continuous cell line, stable cell line, cancer cell line, and the like. In some embodiments, the mammalian cell is isolated or derived from a human subject. In some embodiments, the cell is isolated or derived from a healthy subject. In some embodiments, the cell is isolated or derived from a subject afflicted by a disease or a condition. Non-limiting examples for a disease or condition include but are not limited to, immunodeficiency disease, and cell-proliferation-associated disease.

Methods for isolating cells either from a subject or a culture as mentioned hereinabove are common and would be apparent to one of ordinary skill in the art.

According to some embodiments, the invention is directed to a system comprising multiple types of mammalian cells.

As used herein, the term “multiple” refers to at least 2, at least 3, at least 4, at least 5, at least 6, at least 7, at least 8, at least 9, at least 10, or any range therebetween. In some embodiments, multiple is 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10. Each possibility represents a separate embodiment of the invention.

In some embodiments, there is provided a first type of a mammalian cell.

In some embodiments, the first type of a mammalian cell of the invention is a proliferating cell, a differentiating cell, or both.

In some embodiments, the first type of a mammalian cell is a cell secreting angiogenesis modulating factor(s). In some embodiments, the first type of a mammalian cell initiates, is involved in, propagates, potentiates, or any combination thereof, or any equivalent thereof, angiogenesis, neo-vascularization, or both. In some embodiments, the first type of a mammalian cell is an epithelial cell, an adipocyte, a hepatocyte, or a pancreatic cell. In some embodiments, an epithelial cell is a retinal pigment epithelial cell or a kidney epithelial cell.

As used herein, the terms “proliferating cell” or “proliferation” encompasses any cell undergoing DNA replication and mitosis.

In some embodiments, a proliferating cell divides faster than a normal cell. In some embodiments, the proliferating cell divides more frequently than a normal cell. In some embodiments, the proliferating cell divides faster and more frequently than a normal cell. In some embodiments, the proliferating cell is an abnormally proliferating cell. In some embodiments, the proliferating cell undergoes dysregulated cell cycle. In some embodiments, the proliferating cell has increased amount of DNA compared to control. In some embodiments, the proliferating cell has increased differentiation activity compared to a normal cell. In some embodiments, the proliferating cell is capable of differentiating into the three germ layers (e.g., endoderm, mesoderm, and ectoderm). In some embodiments, the proliferating cell participates in tumorigenesis, promotes tumorigenesis, or both. In some embodiments, the proliferating cell forms teratomas or promotes formation thereof. In some embodiments, the proliferating cell promotes angiogenesis and/or neo-vascularization. In some embodiments, the proliferating cell is a cancerous cell. In some embodiments, the cancerous cell is a solitary cancerous cell. In some embodiments, the cancerous cell is a cell in a tumor. In some embodiments, a portion of the proliferating cells of the present invention are in a form of a tumor. In some embodiments, a portion of the proliferating cells is in a form of a cystoid or a gland. In some embodiments, a portion of the proliferating cells is in a form of any three-dimensional self-organized structure. In some embodiments, at least a portion of the proliferating cells is not in a form of a two-dimensional layer (e.g., a mono- or multiple-layer).

In some embodiments, the proliferating cell or differentiated cell is introduced with an exogenous polynucleotide. In some embodiments, the proliferating cell or differentiated cell is derived from a cell line comprising an exogenous polynucleotide sequence. In some embodiments, the proliferating cell or differentiated cell expresses an exogenous polynucleotide. Non-limiting examples for an exogenous polynucleotide for expression by a proliferating cell or a differentiated cell of the invention include but are not limited to a green fluorescence protein (GFP), a T cell receptor (TCR), and chimeric antigen receptor (CAR). Methods of introducing exogenous polynucleotides and isolating of cells expressing thereof are very common and would be apparent to one of ordinary skill in the art.

As used herein, “a portion” is at least 1%, 2%, 5%, 10%, 20%, 35%, 50%, 70%, 80%, 90%, 99%, or any value or range therebetween. In some embodiments, a portion is 1-5%, 4-15%, 10-25%, 20-45%, 40-70%, 65-85%, or 80-99%. Each possibility represents a separate embodiment of the invention.

In one embodiment, the proliferating cell promotes formation of skin plaque. In one embodiment, the proliferating cell promotes inflammation. In one embodiment, the proliferating cell promotes exfoliation of the skin.

In some embodiments, the proliferating cell is a skin cell. In some embodiments, the skin cell is an abnormal skin cell. In some embodiments, the skin cell is an abnormally proliferating skin cell. In some embodiments, the skin cell is a keratinocyte. In some embodiments, the keratinocyte is a basal keratinocyte. In some embodiments, the keratinocyte is a differentiated suprabasal keratinocyte.

As used herein, the term “normal cell” refers to a control or a naïve cell. In some embodiments, a control cell is a non-proliferating cell. In some embodiments, a control cell is a cell isolated or obtained or derived from a neighboring or adjacent tissue to the tissue from which the proliferating cell was isolated or obtained or derived. In some embodiments, the control cell and the proliferating cell are isolated or obtained or derived from the same tissue, with the control cell being isolated or obtained or derived from a healthy subject (e.g., non-afflicted) or isolated or obtained or derived from a subject before the subject was diagnosed or afflicted by a cell proliferation-associated disease.

In some embodiments, a disease associated with cell proliferation is cancer or pre-malignancy.

In one embodiment, a disease associated with cell proliferation is psoriasis.

The terms “differentiation” and “differentiated” as used herein refers to the cellular development of a cell from a primitive stage to a mature formation that is associated inter alia with the expression of characteristic set of cell surface antigenic markers. In the context of cell ontogeny, the adjective “differentiated”, or “differentiating” is a relative term. A “differentiated cell” is a cell that has progressed further down the developmental pathway than the cell it is being compared with. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (“terminally differentiated cell”).

In some embodiments, the differentiating cell is a stem cell. In some embodiments, the differentiating cell is a progenitor cell.

The term “stem cell” is used herein to refer to a cell (e.g., plant stem cell, vertebrate stem cell) that has the ability both to self-renew and to generate a differentiated cell type (see Morrison et al. (1997) Cell 88:287-298). Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny. “Progenitor cells” differ from stem cells in that they typically do not have the extensive self-renewal capacity.

Non-limiting examples of stem cells include, but are not limited to, an embryonic stem cell, a mesenchymal stem cell, a pluripotent stem cell, an induced pluripotent stem cell, an adipose-derived stem cell, and the like.

In some embodiments, a differentiating cell is capable of differentiating into any cell type of the three germ layers (e.g., endoderm, mesoderm, and ectoderm). In some embodiments, a differentiating cell is a stem cell.

In some embodiments, there is provided a second type of a mammalian cell.

In some embodiments, the second type of a mammalian cell of the invention comprises an immune cell, an endothelial cell, or any progenitor cells thereof.

As used herein, the term “immune cell” encompasses any cell of the immune system. In some embodiments, an immune cell is an infiltrating cell. In some embodiments, an immune cell is a cytokine-mediated remote killing cell. In some embodiments, an immune cell is arresting cell-cycle of a target cell.

In some embodiments, an immune cell is a lymphocyte. In some embodiments, the lymphocyte is an activated lymphocyte. In some embodiments, the lymphocyte is a primed lymphocyte. In some embodiments, the lymphocyte is a differentiated lymphocyte. In some embodiments, the lymphocyte is a cytotoxic lymphocyte. In some embodiments, the lymphocyte is an immunosuppressive lymphocyte. In some embodiments, the lymphocyte is a regulatory lymphocyte. Non-limiting examples of a lymphocyte include but are not limited to, B lymphocyte, T lymphocyte (such as helper, effector, memory, and others), plasma cell, natural killer cell (NK), gamma delta T cell, NK T cell, and any progenitor thereof.

In some embodiments, an immune cell is a myeloid cell. In some embodiments, the myeloid cell is activated. In some embodiments, the activated myeloid cell is classically- or alternatively activated myeloid cell.

Non-limiting examples of a myeloid cell include, but are not limited to, multipotent hematopoietic stem cell, hemocytoblast, common myeloid progenitor cell, megakaryocyte, erythrocyte, mast cell, myeloblast, basophil, neutrophil, eosinophil, monocyte, and a macrophage.

As used herein, the term “endothelial cell” refers to any cell that lines the inner surface of a blood vessel or a lymphatic vessel, which forms an interface between the fluid circulating within in the vessel, e.g., blood or lymph, and the wall of the vessel.

In some embodiments, an endothelial cell is a human umbilical vascular endothelial cell.

As used herein, the term “progenitor cell” encompasses any mesodermal stem cell, an embryonic stem cell (ESc), an adult stem cell, a differentiated ESc, a differentiated adult stem cell, and an induced pluripotent Stem cell (iPSc). As used herein, the term “progenitor cell” refers to a cell capable of giving rise to differentiated cells in multiple lineages, such as, endothelial cells, myoblasts, fibroblasts, adipocytes, stromal cells, fibroblasts, pericytes, and smooth muscle cells. “Progenitor cells” differ from stem cells in that they typically do not have the extensive self-renewal capacity.

As used herein, the terms “infiltrating cell” or “infiltration” refer to a cell attempting to penetrate or penetrating a surface.

In some embodiments, infiltration of a surface encompasses one or more of activities selected from: pressuring, compressing, straining, penetrating, squeezing, pushing, shearing, moving, eroding, and degrading the surface. In some embodiments, a cell performing infiltration activity indicates the cell has high probability of infiltrating a target. In some embodiments, a target is a tissue. In some embodiments, a target is interstices. In some embodiments, a target is a region comprising the first type of a mammalian cell of the invention. Non-limiting examples of a target include, but are not limited to, fat tissue, muscle tissue, skin tissue, a tumor, blood vessel, and lining between cells of a similar type (whether normal or malignant, and others). In some embodiments, the extent of infiltration activity varies among cells of different types or origin. In one embodiment, an infiltrating cell has an increased infiltration activity compared to control, wherein the control comprises a cell of a non-or a low infiltration activity.

In some embodiments, the immune cell of the invention is a cytokine-mediated remote killing cell. As used herein, the term “cytokine-mediated remote killing cell” refers to any cell producing, secreting, or binding to a cytokine, or any combination thereof, which subsequently promotes or results in the killing of a target cell. In some embodiments, the immune cell promotes cell-cycle arrest of a target cell, remotely through cytokine or other signaling molecule, production or release, or any combination thereof.

In some embodiments, the target cell of the immune cell as mentioned hereinabove, is the first type of a mammalian cell of the invention.

### A Mammalian-Avian Chimeric System

According to another embodiment, there is provided an animal model system comprising a chimera between an avian egg's CAM and the hereinabove mentioned mammalian cells. As used herein, the chimeric system comprises an avian egg's CAM, and normal, diseased or genetically transformed mammalian cells, or any combination thereof.

As used herein, the term “CAM” refers to a vascular membrane that can be found in an amniote egg which comprises the chorionic epithelium, and the mesenchyme and allantoic epithelium.

In some embodiments, the present invention is directed to a chimeric system comprising an avian egg. In some embodiments, the egg is a fertilized egg. In some embodiments, the fertilized egg comprises an egg shell. In some embodiments, at least a portion the egg shell has been removed from the fertilized egg. In some embodiments, the fertilized egg comprises an embryo. In some embodiments, the fertilized egg comprises a CAM.

A fertilized egg (comprising an embryo and CAM) of any avian species can be utilized according to the present invention, non-limiting examples of which include, but are not limited to, chicken (Gallus gallus), turkey (Meleagris gallopavo), and duck (Cairina moschata).

As used herein, the chimeric system is simulating an in vivo environment. The term “in vivo” encompasses “in ovo”. In some embodiments, the chimeric system can be used for the testing of drugs. In some embodiments, the chimeric system can be used for the testing of therapeutic compounds (e.g., chemotherapeutics or anti-cancer therapeutics). In some embodiments, the chimeric system can be used for testing communication between multiple cell types. In some embodiments, the chimeric system can be used for testing cell differentiation.

In some embodiments, the chimeric system further comprises one or more stimulatory agents, as disclosed hereinbelow.

In some embodiments, the chimeric system further comprises a therapeutic agent. As used herein, the term “therapeutic agent” refers to any substance having any one of healing, curative, remedial, medicinal, restorative, salubrious, reparative, corrective and beneficial activity, of a subject in need thereof.

Non-limiting examples of a therapeutic agent include but are not limited to, a chemical agent, a biological agent (e.g., a molecule, a macro-molecule, a polypeptide, a cell, and a microorganism), a contact sensitizer, an allergen, a topical cream, a gaseous agent, a pharmaceutical composition, radiation, mechanical ablation, thermal ablation, a chemotherapeutic agent and an anti-cancer agent.

### Method of Preparation

According to another embodiment, there is provided a method for preparing the mammalian-avian chimeric system of the invention, comprising: providing a fertilized avian egg comprising a CAM; grafting a first type of a mammalian cell dispersed in a hydrogel to the CAM; and grafting a second type of a mammalian cell dispersed in a hydrogel to the CAM.

In some embodiments, the mammalian cells of the invention are dispersed in a hydrogel. In some embodiments, the cells are dispersed in the hydrogel before being grafted to the mammalian-avian chimeric system. In some embodiments, the cells are dispersed in the hydrogel before being grafted to the CAM. In some embodiments, the hydrogel comprising the dispersed cells is grafted to the CAM as a liquid (pre-gel composition). In some embodiments, the hydrogel comprising the dispersed cells that is grafted to the CAM as a liquid (i.e., pre-gel composition) solidifies on the CAM (i.e., a gel composition). In some embodiments, the hydrogel comprising the dispersed cells is grafted to the CAM as a semi-solid. In some embodiments, the hydrogel comprising the dispersed cells is grafted to the CAM after being partially gelatinized or jellified. In some embodiments, the hydrogel comprising the dispersed cells is provided to the CAM after being completely gelatinized or jellified. As used herein, the term “partially” encompasses 1-6%, 5-15%, 10-25%, 20-45%, 40-75%, 65-85%, 80-95%, or 90-99%. Each possibility represents a separate embodiment of the invention.

In some embodiments, a hydrogel grafted according to the method of the invention comprises at least 35,000 cells, at least 50,000 cells, at least 75,000 cells, at least 100,000 cells, at least 125,000 cells, at least 150,000 cells, at least 175,000 cells, at least 200,000 cells, at least 250,000 cells, at least 300,000 cells, at least 350,000 cells, at least 375,000 cells, at least 400,000 cells, at least 425,000 cells, at least 450,000 cells, at least 500,000 cells, at least 550,000 cells, at least 625,000 cells, at least 750,000 cells, at least 800,000 cells, at least 850,000 cells, at least 900,000 cells, at least 925,000 cells, at least 950,000 cells, at least 975,000 cells, at least 1,000,000 cells, at least 1,200,000 cells, at least 1,500,000 cells, or any value or range therebetween. In some embodiments, the hydrogel comprises 35,000-50,000 cells, 45,000-70,000 cells, 55,000-85,000 cells, 75,000-100,000 cells, 95,000-150,000 cells, 135,000-185,000 cells, 165,000-200,000 cells, 195,000-250,000 cells, 225,000-350,000 cells, 300,000-375,000 cells, 365,000-450,000 cells, 400,000-485,000 cells, 470,000-550,000 cells, 515,000-650,000 cells, 595,000-625,000 cells, 600,000-650,000 cells, 635,000-695,000 cells, 690,000-750,000 cells, 735,000-775,000 cells, 760,000-850,000 cells, 825,000-890,000 cells, 875,000-950,000 cells, 925,000-950,000 cells, 935,000-990,000 cells, 955,000-1,250,000 cells, 995,000-1,250,000 cells, or 1,200,000-1,500,000 cells. Each possibility represents a separate embodiment of the invention.

In some embodiments, a hydrogel comprising a mammalian cell of the invention is grafted to a fertilized avian egg's CAM on embryonic day 5 (E5), E6, E7, E8, E9, E10, E11, E12, E13, E14, E15, E16, or E17. In some embodiments, the mammalian cell of the invention is grafted to a fertilized avian egg's CAM on E5-E7, E6-E8, E5-E9, E7-E10, E8-E11, E9-E12, E7-E13, E11-E14, E10-E15, E6-E17, E8-16, or E15-E17. Each possibility represents a separate embodiment of the invention.

In some embodiments, a first type of a mammalian cell of the invention is grafted to a fertilized avian egg's CAM on E5, E6, E7, E8, E9, E10, E11, E12, or E13. In some embodiments, the first type of a mammalian cell of the invention is grafted to the fertilized avian egg's CAM on E5-E7, E6-E8, E5-E9, E7-E10, E8-E11, E9-E12, or E10-E13. Each possibility represents a separate embodiment of the invention.

In some embodiments, a second type of a mammalian cell of the invention is grafted to a fertilized avian egg's CAM on E8, E9, E10, E11, E12, E13, E14, E15, E16, or E17. In some embodiments, the second type of a mammalian cell of the invention is grafted to the fertilized avian egg's CAM on E7-E10, E8-E11, E9-E12, E7-E13, E11-E14, E10-E15, E11-E16, or E12-E17. Each possibility represents a separate embodiment of the invention.

In some embodiments, mammalian cells of the invention are dispersed in a hydrogel. The terms “dispersed” and “suspended” are interchangeable.

As used herein, the term “hydrogel” refers to any natural or synthetic organic polymer that can be gelled, or polymerized or solidified (e.g., by aggregation, coagulation, hydrophobic interactions, or cross-linking) into a structure that entraps water and/or other molecules. The terms “hydrogel” and “matrix pellet” are used herein interchangeably.

In some embodiments, the hydrogel comprises naturally occurring substances, such as, fibrinogen, fibrin, thrombin, chitosan, collagen, poly(N-isopropylacrylamide), hyaluronate, albumin, collagen, synthetic polyamino acids, prolamines, polysaccharides such as alginate, heparin, and other naturally occurring biodegradable polymers of sugar units. In some embodiments, the hydrogel is an ionic hydrogel comprising, for example, ionic polysaccharides, such as alginates or chitosan. Ionic hydrogels may be produced by cross-linking the anionic salt of alginic acid, a carbohydrate polymer isolated from seaweed, with ions, such as calcium cations.

Hydrogels disclosed herein, can be used for scaffolds for cell adherence, and are made by any of a variety of techniques known to those skilled in the art. Salt-leaching, porogens, solid-liquid phase separation (sometimes termed freeze-drying), and phase inversion fabrication are used, in some embodiments, to produce porous scaffolds.

In some embodiments, the hydrogel further comprises compounds which promote cell viability, adherence, engraftment, or a combination thereof. In some embodiments, the hydrogel comprises a growth factor. Hydrogels are commercially available and common, and a non-limiting example of which includes Matrigel®, or Cultrex®.

In some embodiments, mammalian cells of the invention are dispersed in a plurality of separate hydrogels. As used herein, “plurality of separate hydrogels” encompasses two or more, three or more, four or more, or five or more separate hydrogels. Each possibility represents a separate embodiment of the invention.

In some embodiments, separate hydrogels are grafted to the CAM at a distance which enables secretion of factors from one hydrogel to the other, and vice versa. In some embodiments, separate hydrogels are grafted to the CAM at a distance which enables secretion of factors from one hydrogel through the CAM vasculature system to the other hydrogel, and vice versa. In some embodiments, separate hydrogels are grafted to the CAM at a distance which enables one cell population engrafted in one hydrogel to affect, infiltrate, induce, inhibit, communicate, or any combination thereof, with the other cell population engrafted in the other hydrogel, or vice versa. In some embodiments, the method of the present invention further comprises a step of determining a suitable distance to engraft the separate hydrogels so as to enable communication between the cells engraft in each of the aforementioned hydrogels, wherein the communication is direct communication, such as cell infiltration, or indirect communication, such as secretion of factors through the hydrogels, through the CAM's vasculature system, or both. In some embodiments, communication comprises secretion of factors stimulating: activity of the other cell type, e.g., cytokines, and angiogenic molecules, cell cytotoxicity, apoptosis, differentiation, susceptibility to toxic agents, such as chemotherapeutic compounds, cell infiltration (such as performed by immune cells), or any combination thereof.

In some embodiments, separate hydrogels are grafted to the CAM at a distance of not more than 1 mm from one another, not more than 2 mm from one another, not more than 3 mm from one another, not more than 4 mm from one another, not more than 5 mm from one another, not more than 6 mm from one another, not more than 7 mm from one another, not more than 8 mm from one another, not more than 9 mm from one another, not more than 10 mm from one another, or any range or value therebetween. In some embodiments, separate hydrogels are grafted to the CAM at a distance of 0.1-2 mm from one another, 1-3 mm from one another, 2.5-4 mm from one another, 3-5 mm from one another, 2-6 mm from one another, 4-7 mm from one another, 5-8 mm from one another, 4-9 mm from one another, or 5-10 mm from one another. In some embodiments, separate hydrogels are grafted to the CAM at a distance of at least 0.1 mm from one another. Each possibility represents a separate embodiment of the invention.

In some embodiments, hydrogels grafted separately to the CAM according to the method of the invention can fuse to one another. In some embodiments, hydrogels grafted separately to the CAM fuse and form a single mass comprising multiple conjoined (or fused) hydrogels. As used herein, the fused hydrogel comprises two or more types of mammalian cells of the invention. In some embodiments, the fused hydrogel is contained within a tumor. In some embodiments, a tumor comprising the fused hydrogel, further comprises chick blood vessels, fibroblasts, epithelial cells, or any combination thereof.

As used herein, the fused hydrogel comprises two side A, a partition, and side B. As used herein, “side A”, and “side B” refer to the locations wherein initially each one of the two cell population was suspended. In some embodiments, at least one cell suspended within side A infiltrates or migrates to side B. In some embodiments, at least one cell suspended within side B infiltrates or migrates to side A.

The term “partition” refers to the interphase formed between side A and side B when the herein disclosed separate hydrogels fuse.

In some embodiments, separate hydrogels are grafted simultaneously to the system of the invention.

In some embodiments, separate hydrogels are grafted sequentially to the system of the invention. In some embodiments, sequentially is grafting of the first type of a mammalian cell followed by grafting of the second type of a mammalian cell. In some embodiments, sequentially is grafting of the second type of a mammalian cell followed by grafting of the first type of a mammalian cell.

In some embodiments, separate hydrogels are grafted sequentially so as to provide particular microenvironment required for one type of cell engrafted in one hydrogel, or for both types of cells. In some embodiments, the method of the present invention further comprises a step of determining a suitable time for engrafting one type of cell engrafted in one hydrogel, or both types of cells, wherein the suitable time is with respect to the embryonic day of the CAM. In some embodiments, the method of the present invention further comprises a step of determining a suitable time for engrafting one type of cell engrafted in one hydrogel, or both types of cells, wherein the suitable time is with respect to the establishment or formation of microenvironments adequate for the growth, functionality, communication, or any combination thereof, of these types of cells.

In some embodiments, the method comprises a step of determining the formation of vasculature system sufficient so as to screen therapeutic compounds as described hereinbelow. Vasculature system sufficiency so as to screen therapeutic compounds is referred to hereinbelow as the “angiogenesis threshold”. In some embodiments, the method comprises engraftment of hydrogels comprising mammalian cells, and determining the level of angiogenesis thereafter. There is need to determine a threshold level of angiogenesis in order to examine or screen for therapeutic compounds. In some embodiments, the method comprises a step of determining the aforementioned angiogenesis threshold. In some embodiments, the method for screening or examining the activity of a therapeutic compound is performed or commences after the system has been determined to pass or meet the angiogenesis threshold.

In some embodiments, sequentially grafting comprises 1-6 hours between each grafting, 5-15 hours between each grafting, 10-18 hours between each grafting, 16-24 hours between each grafting, 20-36 hours between each grafting, 30-48 hours between each grafting, 40-56 hours between each grafting, 50-72 hours between each grafting, 70-85 hours between each grafting, 80-100 hours between each grafting, or 95-120 hours between each grafting. In some embodiments, sequentially grafting comprises 1 hour at most between each grafting, 6 hours at most between each grafting, 8 hours at most between each grafting, 12 hours at most between each grafting, 16 hours at most between each grafting, 24 hours at most between each grafting, 36 hours at most between each grafting, 48 hours at most between each grafting, 60 hours at most between each grafting, 72 hours at most between each grafting, 84 hours at most between each grafting, 96 hours at most between each grafting, 120 hours at most between each grafting, or any value or range therebetween. Each possibility represents a separate embodiment of the invention.

In some embodiments, the provided method further comprises a step of determining the engraftment of the first type of a mammalian cell to the CAM. In some embodiments, determining the engraftment of the first type of a mammalian cell comprises determining any activity selected from: cell proliferation, cell growth, cell survival, tumorigenesis, angiogenesis, and neo-vascularization. Methods for determining any of the abovementioned activities are common and would be apparent to one of ordinary skill in the art. Non-limiting examples include, but are not limited to, apoptosis assay, metabolic stains or assays (e.g., MTT, XTT assays), viability stains (acridine orange, and trypan blue), immunohistochemistry using specific proliferation markers (e.g., Ki67) or angiogenic markers (e.g., CD31, CD105, VEGF, etc.), flow cytometry (e.g., DNA stains for cell cycle analysis, such as Hoechst), and light microscopy.

In some embodiments, the method further comprises contacting the mammalian cells of the invention with one or more stimulatory agents. As used herein, the term “stimulatory agent” encompasses any compound, molecule, or agent which is capable of provoking a target cell (i.e., a mammalian cell of the invention), wherein provoking results in increasing cellular activity of the target cell. Non-limiting examples for cellular activity include, but are not limited to, gene expression (RNA transcription and protein translation), differentiation, cell migration, tissue infiltration, cytotoxicity, cytokine release, granule release, proliferation, neoplastic transformation, drug-resistance, protein secretion, DNA duplication, and others. Methods of detecting alterations of cellular activities, as described hereinabove would be apparent to a skilled artisan. Non-limiting examples include, but are not limited to, microscopy, polymerase chain reaction (quantitative and/or qualitative), enzyme linked immunosorbent assay (e.g., direct ELISA, sandwich ELISA), immunohistochemistry or immunocytochemistry, flow cytometry, hybridization assays (e.g., RNA in situ hybridization, northern-blot), and others.

In some embodiments, one or more stimulatory agents comprises 2 or more, 3 or more, 4 or more, 5 or more, 6 or more, 7 or more, 8 or more, 9 or more, or 10 or more stimulatory agents. In some embodiments, one or more stimulatory agents comprises 1-4, 2-5, 3-4, 4-7, 2-6, 5-8, 6-9, or 7-10 stimulatory agents. Each possibility represents a separate embodiment of the invention.

In some embodiments, the stimulatory agent increases a cellular activity by at least 5%, by at least 25%, by at least 50%, by at least 100%, by at least 200%, by at least 300%, by at least 400%, by at least 500%, by at least 750%, or by at least 1,000% compared to a baseline in which the stimulatory agent is not present, or any value or range therebetween. In some embodiments, the stimulatory agent increases a cellular activity by 5-50%, 25-100%, 75-200%, 175-300%, 250-400%, 350-500%, 475-750%, or 700-1,000% compared to a baseline in which the stimulatory agent is not present. Each possibility represents a separate embodiment of the invention.

In some embodiments, the stimulatory agent is provided to the first type of a mammalian cell. In some embodiments, the stimulatory agent is provided to the second type of a mammalian cell. In some embodiments, the stimulatory agent is provided to the first type of a mammalian cell and the second type of a mammalian cell. In some embodiments, the stimulatory agent is provided to the first type of a mammalian cell before engraftment to the CAM. In some embodiments, the stimulatory agent is provided to the first type of mammalian cell in the hydrogel prior to engraftment. In some embodiments, the stimulatory agent is provided to the first type of a mammalian cell after engraftment to the CAM. In some embodiments, the stimulatory agent is provided to the second type of a mammalian cell before engraftment to the CAM. In some embodiments, the stimulatory agent is provided to the second type of mammalian cell in the hydrogel prior to engraftment. In some embodiments, the stimulatory agent is provided to the second type of a mammalian cell after engraftment to the CAM. In some embodiments, the stimulatory agent is provided to the first type of a mammalian cell and the second type of a mammalian cell before engraftment to the CAM. In some embodiments, the stimulatory agent is provided to the first type of mammalian cell and the second type of a mammalian cell in the hydrogel prior to engraftment. In some embodiments, the stimulatory agent is provided to the first type of a mammalian cell and the second type of a mammalian cell after engraftment to the CAM. In some embodiments, the stimulatory agent is provided to the first type of a mammalian cell before engraftment to the CAM and to the second type of a mammalian cell after engraftment to the CAM. In some embodiments, the stimulatory agent is provided to the first type of a mammalian cell after engraftment to the CAM and to the second type of a mammalian cell before engraftment to the CAM.

In some embodiments, providing the stimulatory agent comprises contacting the mammalian cell of the invention with the stimulatory agent. In some embodiments, providing the stimulatory agent comprises intravenously injecting the stimulatory agent into the CAM blood vessels.

In some embodiments, the provided method further comprises a step of determining the effector activity of the second type of a mammalian cell.

In some embodiments, effector activity is selected from: infiltration, cytokine secretion, cytokine-mediated remote killing, cell-cycle arresting, angiogenesis, and neo-vascularization. Methods for determining infiltration or presence of cells from one type to hydrogel comprising another type of cells would be apparent to one of ordinary skill in the art. A non-limiting example includes immunohistochemistry comprising specific markers for each type of cell assays, as exemplified hereinbelow (e.g., a marker for an infiltrating cell, such as CD45, and CD56), an angiogenic marker (e.g., CD31), and a marker of a proliferating cell, such as MelanA). Methods for cytokine assaying (e.g., quantification and functionality), are well known in the art, and non-limiting examples of which include but are not limited to multiple types of ELISA, immunohistochemistry, T cell activation assay, and others. Methods for determining cell cycle arrest are common and would be apparent to one of ordinary skill in the art. Non-limiting examples, include but are not limited to, cell-cycle assay kits and flow cytometry (such as by Abcam, Thermo Scientific, etc.), and immunohistochemistry (e.g., Ki67, BrdU incorporation).

In some embodiments, the first type of a mammalian cell and the second type of a mammalian cell are obtained, derived or isolated from different mammalian species, as mentioned hereinabove. As a non-limiting example, the first type of a mammalian cell is a human cell (e.g., proliferating cell) and second type of a mammalian cell is a murine cell (e.g., immune cell).

The herein disclosed system, in some embodiments thereof, is directed to an in vitro study model comprising a vasculature system. The herein disclosed system, in some embodiments thereof, is suitable for studying cell-cell communication, screening for therapeutic agents, screening for cell-cell communication agents, or any combination thereof.

### Method of Screening

According to another embodiment, there is provided a method of screening for a therapeutic compound for treating a disease associated with cell proliferation, comprising contacting the mammalian cells engrafted to an avian egg's CAM with a compound, and determining the effect of the compound on the mammalian cells.

In one embodiment, the compound is a drug for chemotherapy. In another embodiment, the compound is a drug is for anti-cancer therapy. In another embodiment, the compound is a drug for chemotherapy and anti-cancer therapy.

In some embodiments, the method comprises providing the mammalian-avian chimeric model system of the invention; and contacting the system with a compound. In some embodiments, the method comprises determining a change of a phenotype in the first type of a mammalian cell in the presence of the compound. In some embodiments, the method comprises determining the phenotype in the first type of a mammalian cell in the absence of the compound. In one embodiment, the phenotype in the first type of a mammalian cell in the absence of the compound is regarded as control or baseline.

In some embodiments, the change of a phenotype in the first type of a mammalian cell in the presence of the compound compared to the control or baseline is indicative of the suitability of the compound in treating the disease associated with cell proliferation.

As used herein, the term “change” encompasses an increase or a decrease.

In some embodiments, the change is by at least 5%, at least 25%, at least 50%, at least 100%, at least 200%, at least 300%, at least 400%, at least 500%, at least 750%, or at least 1,000% change compared to the control or baseline, or any value or range therebetween. In some embodiments, the change is 5-50%, 25-100%, 75-200%, 175-300%, 250-400%, 350-500%, 475-750%, or 700-1,000% change compared to the control or baseline. Each possibility represents a separate embodiment of the invention.

As used herein, the term “phenotype” encompasses any cellular process involving gene and/or protein expression which affects and/or determines the cell's particular morphology, function, communication, or any combination thereof. In some embodiments, cellular phenotype comprises proliferation rate, cell death rate (such as apoptosis or necrosis), differentiation, tumorigenesis, metabolic rate, cytotoxicity, or any combination thereof.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Example 1

### Engraftment of a Small Number of Cancer Cells

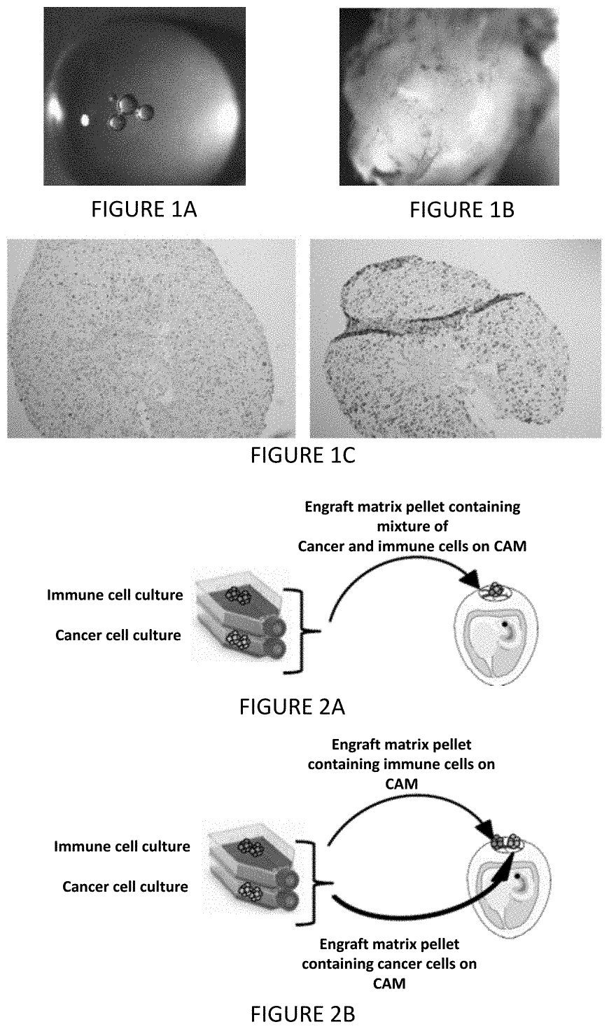

The ovarian cancer cell line PEA2 was embedded in a matrix pellet by resuspending 3×10⁵ PEA2 cells in 15 μl Matrigel®, on ice, and pipetting onto a solid surface (Parafilm) at 37° C. for 30 minutes (FIG. 1A). The Matrigel® pellet that formed was then placed on the CAM of an E9 egg, close to an underlying CAM blood vessel junction. It was not necessary to wound the CAM surface blood vessels in order to achieve a high rate of engraftment. The engrafted PEA2-containing matrix pellet (FIG. 1A) formed a tumor (diameter ˜5 mm) on the CAM (FIG. 1B). In the process of engraftment, the CAM engulfed the cancer cell matrix pellet and chick blood vessels sprouted new networks throughout the tumor (which were visible on the surface of the excised tumor). Tumors comprising ovarian cancer cells were excised at day E16, fixed in paraformaldehyde and embedded in paraffin blocks. Tissue sections (7 μm thick) were cut and analyzed by histology and immunohistochemistry for cell viability, proliferation and death (FIG. 1C). Tissue sections stained with H&E (hematoxylin and eosin), revealed that most cells looked normal and healthy within the tumor (FIG. 1C, left). Tissue sections were incubated with a mouse antibody to CD44 (which does not bind the chick cells) and stained using a colorimetric anti-mouse IgG-HRP (horseradish peroxidase) enzymatic reaction with the substrate DAB (3,3′Diaminobenzidine, and counterstained with hematoxylin; FIG. 1C, right). While dying cells are known to rapidly lose CD44 expression, the cancer cells in the tumor clearly showed strong CD44 expression, therefore indicating their vitality (FIG. 1C, right).

The inventors concluded that tumors engrafted by the matrix pellet method contained mostly viable cells throughout the tumor mass, in nearly all tumors. Thus, when using this method of engrafting a cell suspension in a solidified Matrigel® or Cultrex® (BME3) pellet, cancer cells can be reliably engrafted into the egg and readily analyzed by standard histology analytical techniques (e.g., immunohistochemistry).

The inventors further engrafted 5×10⁴ to 3×10⁵ melanoma cells (cell line Mel-624) in E8, E9 or E10 eggs. Tumors formed for all cell numbers tested, with a high engraftment rate (>80%), with engraftment on E8 or E9 having the highest engraftment rates.

### Example 2

### Co-Engraftment of Two Types of Foreign Cells

To generate a chimeric egg containing more than one foreign cell type, the inventors have designed several permutations of methods which are presented herein below.

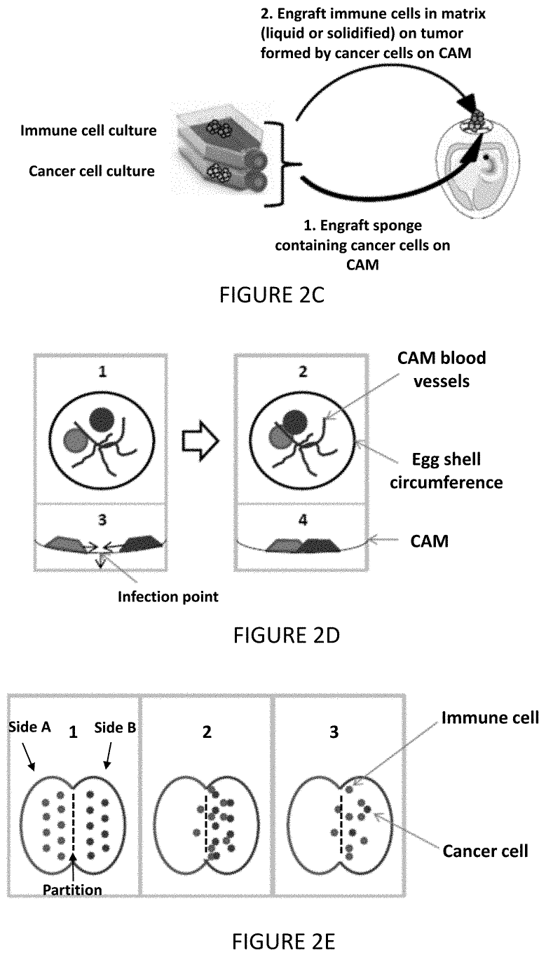

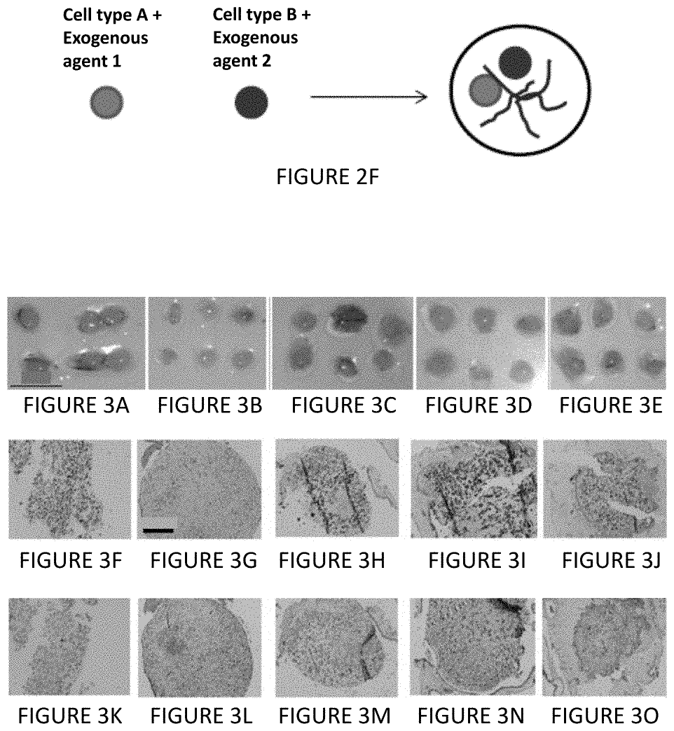

Cancer cells are mixed with immune cells in the same matrix pellet and are then engrafted on the CAM. The two cell types (cancer cell and immune cell) do not come in to immediate direct contact with one another upon matrix pellet solidification, yet subsequently make direct contact with each other over the 1-3 days following matrix pellet engraftment, depending on the level of motility of the cells. The two cell types share a common microenvironment (FIG. 2A).

Two or more cell types (e.g., both cancer cells and foreign immune cells) are engrafted separately in two matrix pellets on the same CAM in proximity one to the other. The two individual matrix pellets induce the formation of two separate tumors on the CAM (FIG. 2B). Alternatively, the matrix pellet comprising the immune cells is engrafted after a tumor has been formed by the cancer cells (FIG. 2C).

The two matrix pellets co-engrafted on the same CAM (FIG. 2B), draw close to one another and eventually fuse to form a single tumor (FIG. 2D).

A tumor can be formed from the fusion of two separate tumors, such that the individual foreign cells from the two sources: (1) do not mix, (2) one cell type invades the other matrix pellet but does not kill the cells resident in the second matrix pellet, or (3) one cell type invades the other matrix pellet and kills the cells resident in the second matrix pellet (FIG. 2E). A fused tumor comprising two foreign cell types is exemplified with immune cells and cancer cells (FIG. 2E).

Alternatively, two separate matrix pellets each comprising one type of cells (e.g., A and B) further comprise exogenous agents (FIG. 2F). The two pellets comprising a suspension of cells mixed with exogenous agents are then engrafted on the CAM of the same egg. While in one matrix pellet cell type A is exposed to exogenous agent 1, in the second matrix pellet cell type B is exposed to exogenous agent 2. The local effect of an exogenous agent acting only on one cell type during the period of engraftment may induce a cellular phenotype, such as immunosuppression or activation of immune cells.

### Example 3

### “Winn” Assay for Measuring Anti-Cancer Cytotoxicity of Human Immune Cells

Matrix (Cultrex® (BME3)) pellets containing a mixture of melanoma cells and TILs were engrafted in the egg and scored for tumor formation. When Mel-624 cells were engrafted alone on the egg CAM, tumors were formed within three days and continued to grow in size (FIG. 3A). Haplotype matched patient TILs (HLA-A2 positive, i.e., TILs 463) from melanoma were activated and rapidly expanded in vitro and resuspended in matrix (3×10⁵ cells per 7.5 μl Cultrex®). Two very common melanoma epitopes that are presented selectively by HLA-A2, are derived from MART-1 (MelanA) and gp-100 (in approximately 1-5% of the TILs population) and are endogenously expressed by Mel-624. Patient TILs recognizing HLA-A2 bound to a gp100 peptide were enriched using HLA-tetramer-epitope selection, and thus were highly likely to recognize Mel-624 cells and attack them. When the TILs/matrix suspension was mixed with the Mel-624 cells/matrix suspension (7.5 μl of each; volumes tested were 7.5 μl, 10 μl, and 15 μl, of each—all giving equivalent results), and left to solidify as a pellet in vitro at 37° C. for 30 minutes, all engrafted pellets either failed to form large tumors or formed tumors that subsequently dried up on the CAM (FIG. 3B). Out of the 12 eggs engrafted with Mel-624 pellets, 9 formed tumors with an average diameter of ˜5 mm, while three failed to form a visible tumor, 96 hours following engraftment (engraftment rate 75%, table 1). When pellets were made with a mix of TILs and Mel-624 cells, out of the 20 eggs engrafted, only 6 formed tumors with an average diameter <3 mm, and the remainder failed to form visible tumors (engraftment rate 30%, table 1).

TABLE 1
Quantification of “Winn” assay comparing
tumors formed by Mel-624 and Mel-624 mixed with
TILs 463, after 4 days incubation following engraftment.

| | M-624 | M-624 + TILs 463 |
|---|---|---|
| Viable tumors (n/total) | 9/12 | 6/20 |
| % | 75% | 30% |

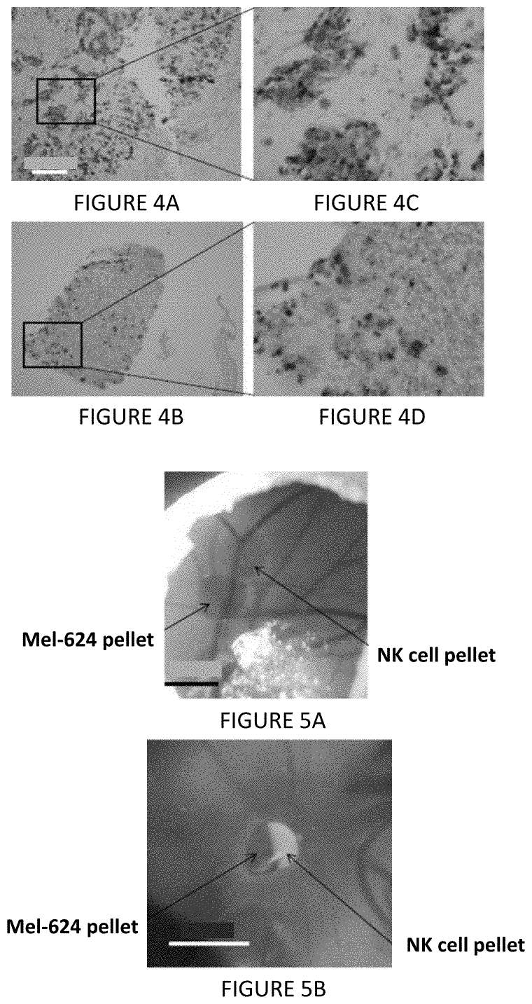

Microscopic histological analysis of fixed tumor tissue sections immunoreacted with antibodies detecting the proliferation marker Ki67 (FIG. 3F-3J) revealed that while in Mel-624 tumors most cells were actively cycling (FIG. 3F), in the TILs-treated tumors there were very few living cycling melanoma cells (FIG. 3G). Immunohistochemistry with an antibody detecting cleaved Caspase 3, an indicator of apoptosis (FIG. 3K-3O), revealed that while very a few cells in Mel-624-only tumors are dying, many Mel-624 cells in the TIL-treated tumors were dying (FIGS. 3F and 3G). Closer inspection of staining by two antibodies, to detect TILs on the one hand (CD8), and all human cells (melanoma and TILs) on the other hand (LAMP1), revealed that TILs surround melanoma cells undergoing apoptosis (FIG. 4).

In parallel, similar experiments were conducted using TILs from an HLA-A2 negative patient (TILs 425), mixed with Mel-624 cells in a Cultrex® (BME3) pellet. These TILs lacked a T cell receptor (TCR) that recognizes the HLA-A2/MART-1 or gp-100 complex which is present on Mel-624, (although it is possible that the TILs recognize other HLA haplotype epitope complexes on the Mel-624 cells). Large tumors were formed in most eggs (FIG. 3E), and immunohistochemical detection revealed that the melanoma cells were proliferating (FIG. 3J) with very little apoptosis (FIG. 3O). Similarly, when TILs recognizing HLA-A2/gp100 and killing Mel-624 cells (TILs 463) were engrafted in a Winn assay with Mel-888 (an HLA-A2 negative melanoma cell line), there was only mild elevation in the number of dying cancer cells (cleaved Caspase 3; FIG. 3N), while proliferation remained high (Ki67; FIG. 3I). Once again, TILs were found in close proximity to the cancer cells, which may indicate that there was not a strong activation of the TILs by Mel-888, possibly due to HLA/epitope complex—TCR mismatch.

### Example 4

### Adjacent Immune Cell Pellet Assay

The inventors then generated fusion tumors whereby two different and separate cell types were brought into interaction with each other, within a tumor, following an engraftment period of 1-2 days.

### NK Cells

Two separate sets of matrix pellets (comprising 15 μl Matrigel®) were prepared from cell suspensions of melanoma cells (Mel-624) and from healthy human donor NK cells, activated in vitro by incubation with a cytokine cocktail on a feeder cell layer. Pellets comprising 3×10⁵ cells were placed on the CAM of day E9 eggs as follows: one Mel-624 pellet was placed on the CAM, and then a pellet of NKs was placed on the same CAM at a distance of <1 mm from the Mel-624 pellet (FIG. 5A). After 2 days (E11) all tumors were found to be half “orange” (the color of the CAM underlying the transparent melanoma cells) and half “white” (the color of the NK cells; FIG. 5B), indicating that fusion of the two matrix pellets occurred with complete efficacy.

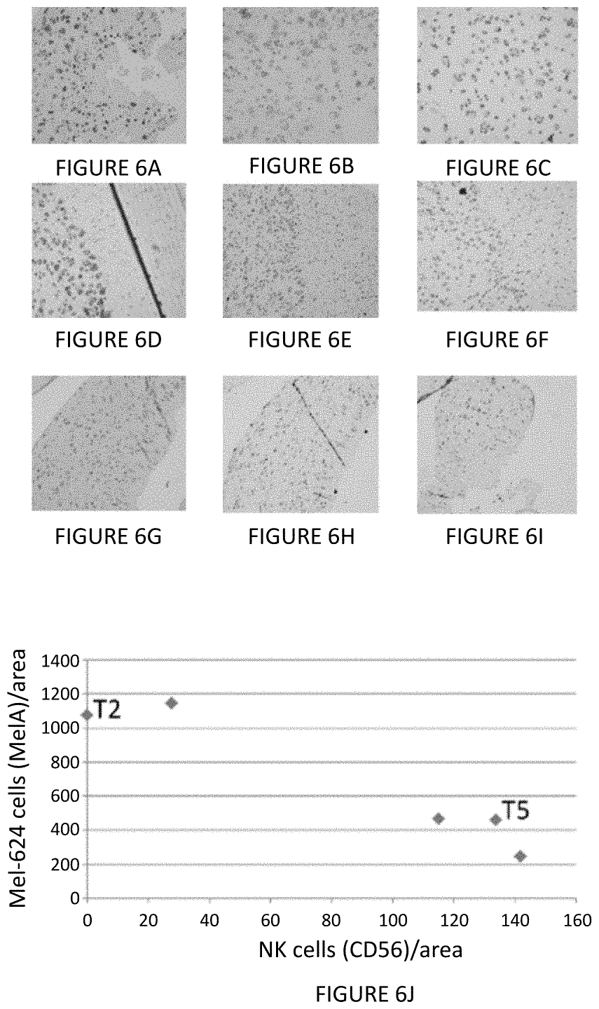

Histological analysis proceeded by immunohistochemistry (IHC) targeted at either MelanA or cleaved Caspase 3 of the fusion tumors revealed that the two Cultrex® (BME3) pellets had indeed juxtaposed to form one single moiety (FIGS. 6D and 6E). Melanoma cells which are significantly larger than the NK cells stained for MelanA, while the latter did not (FIGS. 6D and 6E). In this tumor the NK and Mel-624 cells have not mixed within the tumor. By comparison in a different tumor from the same experiment the NK cells were found throughout the regions of the Mel-624 Cultrex® (BME3) pellet within the tumor (FIG. 611; an NK cell is indicated by an arrow). NK cell infiltration into regions of the tumor occupied by the Mel-624 cells appeared to result in apoptosis of the melanoma cells, as is shown by cleaved Caspase-3 staining by IHC (FIG. 6G). By contrast, Mel-624 did not stain for cleaved Caspase-3 in tumors that do not contain NK cells (FIG. 6C), or in a fusion tumor where the NK cells had not infiltrated the region of the tumor where the melanoma cells reside (FIG. 6F). An inverse correlation was also observed between melanoma cell density and NK cell infiltration, whereby when more than 100 CD56-stained NK cells were observed per imaged field, melanoma cells number was reduced by approximately 50% (FIG. 6J), indicating a significant inhibitory effect on tumor growth.

The inventors further noted, that under these pellet fusion conditions no CAM membrane was observed between the two pellets. Thus, while fusion of the two pellets always results in a tumor containing both pellets juxtaposed to each other, invasion of cells from one pellet to another did not occur in all cases, and this was not due to a physical barrier set by the CAM (FIGS. 6D-6F).

### Tumor Infiltrating Lymphocytes (TILs)

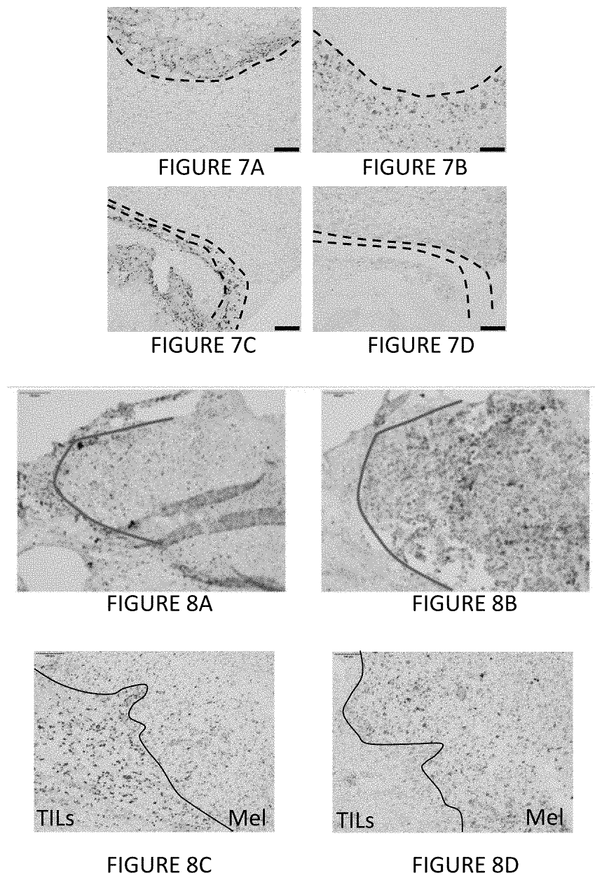

Two separate sets of matrix pellets (comprising 15 μl Matrigel®) were prepared from cell suspensions of melanoma cells (Mel-624) and from TILs. TILs from two different patients (both haplotype HLA-A2) were expanded in vitro and contained cytotoxic T cells reactive to the cell lines Mel-624 and Mel-526 (but not to HLA-A2 negative cell line Mel-888), as revealed by increased IFN-γ secretion. These TILs were not selected for recognition of the HLA tetramer-MART1 and/or gp100 peptide complexes, and thus only 1-5% of the TILs were expected to target Mel-624 or Mel-526 cells. One pellet comprising 3×10⁵ Mel-624 cells was placed on the CAM of a day E9 egg, and a pellet of 3×10⁵ TILs from either patient, was placed on the same CAM at a distance of <1 mm from the Mel-624 pellet (in the manner exemplified in FIG. 5A). All paired pellets fused to form one tumor and upon histological analysis immunostaining revealed distinct compartments of CD45 positive cells (i.e., human immune cells) adjacent to melanoma cells (anti-MelanA antibodies). Two different outcomes were observed for the two sets of TILs from the two patients. TILs from the first patient did not infiltrate the region of the tumor containing the melanoma cells (Mel-624) (FIG. 7), while TILs from the second patient did (FIG. 8).

Control Experiments with Cell Lines

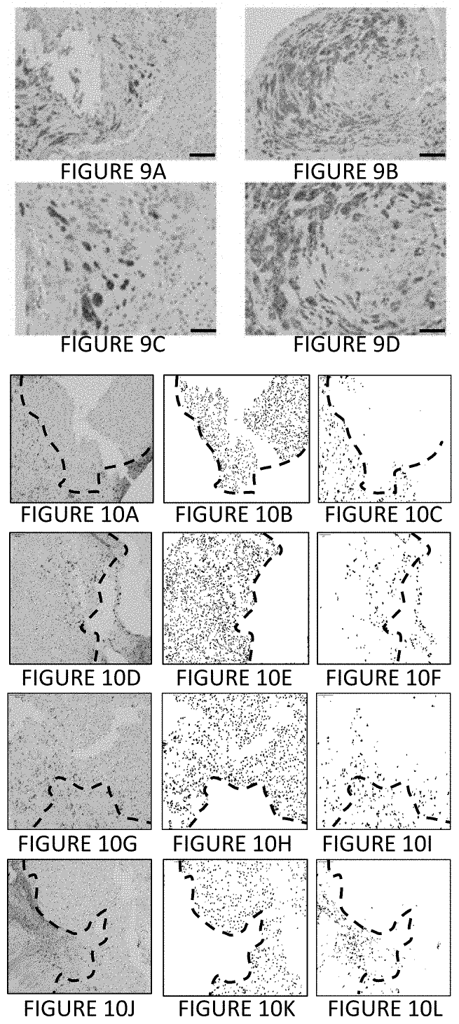

The inventors examined additional pairs of cell types in a similar set of experiments and assayed for invasion of one cell type into the adjacent cell type compartment. Eggs were engrafted with two matrix pellets (one of each cell type) each in 15 μl Matrigel® and placed on the CAM on day E9, within 1 mm distance of each other. Where both matrix pellets in an egg were successfully engrafted, all tumors underwent fusion (100%) for all cell types. Four days (96 h) after engraftment (E13) the tumors were excised, fixed, embedded in paraffin and sectioned for immunohistochemistry. Two combinations of cell pairs, Jurkat and Mel-624 (FIGS. 9A and 9C), and Jurkat and B16 (FIGS. 9B and 9D), were examined. The Jurkat cells mixed with the second cell type though without breaking of as invading individual cells. Inversely, neither B16 nor Mel-624 showed significant invasion of single cells into the Jurkat compartment (FIG. 9; positively stained for CD3).

### Example 5

### Matrix Density and Composition Influences Infiltration of Immune Cells

### Method

Mixtures of Matrigel®, PBS, and human serum in varying proportions were used to resuspend cells—either cancer cells (melanoma cell line Mel-624) or immune cells (PBMCs or TILs). PBMC were harvested from donor whole blood as follows: PBS was added to the blood sample, in ratio of 1:1. Ficoll was added in ratio of 3 (Ficoll): 4 (blood in PBS). The sample was centrifuged for 20-30 min at room temp, 400 g, without brake. Pellet was washed with PBS (×6 volume). Second centrifugation was performed as follows: 15 min, 400 g, at room temp, with brake. Pellet was washed with 7 ml PBS. Sample was centrifuged at room temp for 10 min, 60-100 g.

TILs were from donor #3 (enriched for recognition of HLA A2—gp100 and expanded in vitro with IL-2 and anti-CD3/28 antibody mediated activation).

Recombinant human (rh) IL-2 was added to Matrigel® of side B at concentration 50,000 units/ml; cytokines IP10, SDF-1 and CCL-19 (dissolved in water) were added to side A at an amount of 12 ng each, per egg.

Hereinbelow are tables of matrix components and their proportion (% of volume) in each matrix pellet, and infiltration score.

TABLE 2
Comparison between Matrigel ® and Matrigel ®/human
serum mix, on PBMC infiltration.

| | | | | | Infiltration score | |
|---|---|---|---|---|---|---|
| | “Side A” - cancer | | “Side B” - immune | | Average no. | |
| | cells (Mel-624) | | cells (PBMCs) | | CD45 cells - | # |
| Matrix | | Human | | Human | side A | tumors |
| component | Matrigel ® | serum | Matrigel ® | serum | (±SEM) | scored |
| Group 1 | 100 | 0 | 83 | 0 | 10 ± 5 | 3 |
| Group 2 | 50 | 50 | 50 | 50 | 71 ± 21* | 4 |

*Student T test P value = 0.04

TABLE 3
Comparison between Matrigel ® and Matrigel ®/PBS mix, on TIL infiltration.

| | | | | | | | Infiltration score | |
|---|---|---|---|---|---|---|---|---|
| | “Side A” - cancer cells | | | “Side B” - immune cells | | | Average no. | |
| | (Mel-624) | | | (TIL#3) | | | CD45 cells - | # |
| Matrix | | | Human | | | Human | side A | tumors |
| component | Matrigel ® | PBS | Serum | Matrigel ® | PBS | serum | (±SEM) | scored |
| Group 3 | 50 | 0 | 50 | 50 | 0 | 50 | 64 ± 13 | 7 |
| Group4 | 50 | 50 | | 50 | 50 | 0 | 53 ± 31 | 2** |

**PBS in pellets greatly reduces tumor engraftment (~20% compared to 80% in Groups 1, 2, or 3)

### Results

In an experiment using freshly harvested PBMC, two experimental groups were compared, one in which pellets of Side A and Side B both comprised 100% Matrigel® (Group 1) and one in which both pellets comprised 50% Matrigel® and 50% human serum (Group 2). In Group 1, six tumors were harvested and processed for IHC analysis, of which three showed a partition between Side A and Side B, and two showed low numbers of infiltrating T cells. In Group 2, of six tumors analyzed, four showed a clear partition between Side A and Side B, wherein all four tumors exhibited infiltration by PBMC into Side A in significantly greater numbers (Group 1, Table 1 and FIG. 10).

In a separate experiment using rapidly expanded TIL (from donor #3), either human serum or PBS were mixed with Matrigel® in both Side A and Side B, and infiltration was compared. In Group 3, eleven tumors were harvested and processed for IHC analysis, of which seven showed a partition between Side A and Side B, and six showed high numbers of infiltrating T cells (Table 2, FIG. 10). In Group 4, very few tumors engrafted when PBS was mixed with the Matrigel®, however, five tumors were harvested, three of which showed a clear partition between Side A and Side B, and all three tumors exhibited infiltration by TILs into Side A in similar numbers as those seen in Group 3 (Group 4, Table 2 and FIG. 10).

The comparative effect of mixing PBS or human serum with Matrigel® was shown to be of significant importance, whereby human serum had increased viability of the immune cells, while PBS had reduced the overall engraftment efficacy.

### Conclusions

Reducing Matrigel® content by replacement with PBS greatly reduced tumor viability, yet did not impinge on TILs infiltration activity. When human serum was used to dilute Matrigel®, both cancer cells and immune cells were more viable and engraft well, and thus may greatly increase tumor infiltration by immune cells.

### Example 6

### Discerning Different Infiltration, Cancer Cell Recognition, and Killing Activities of Immune Cells

### Method

Immune cells of different types and origins were compared for their tumor infiltration activity and cancer cell killing properties, as follows. A mixture of Matrigel® and human serum, recombinant human IL-2 (for immune cells, side B) or SDF-1, and IP-10 dissolved in saline (for cancer cells, side A), were used to resuspend cells—either melanoma cell line Mel-624 (or BT474 breast cancer cells, for CAR-T experiment) or immune cells (TILs from patients #2, #3, #4, PBMCs from a donor, or CAR-T cells). Components were mixed cold and then added to a cell pellet (final volume per pellet 15 μl, with side A receiving 300,000 cancer cells per matrix pellet, and side B receiving 900,000 immune cells per matrix pellet). Matrix pellets were formed by dripping 15 μl of the matrix components mixed with cells onto a sterile Parafilm surface and placed in a humidified incubator at 37° C. for 30 minutes to allow solidifying.

Matrix pellet pairs (one “side A” and one “side B”) were placed adjacently (˜1 mm gap between matrix pellet rims) on the CAM of E9 eggs, sealed and returned to the egg incubator. For TILs and PBMCs experiments, 24 h later eggs were unsealed and rhIL-2 (6,000 units in 10 μl RPMI medium) was dripped on the CAM at a distance of 5 mm from the engrafted matrix pellet pairs, and eggs were resealed and returned to egg incubator.

At E13 (96 h after engraftment), tumors were harvested by excision from the egg, rinsed in PBS, fixed in 4% (w/v) paraformaldehyde for 30 minutes, rinsed in PBS and transferred to 70% (v/v) ethanol prior to embedding in paraffin by standard techniques.

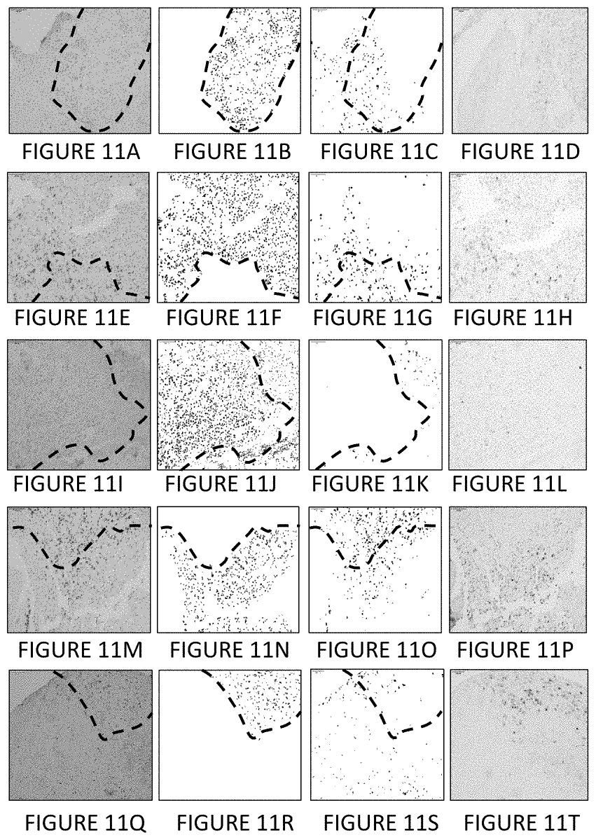

Tumors embedded in paraffin were sectioned in a microtome (6 μm thickness) and stained by immunohistochemistry (IHC) for markers. CD45 was used to detect human immune cells, melanoma cells Mel-624 and breast cancer cells BT474 were identified by morphology and nuclear size (stained with Hematoxylin) and gp100 or CD155 IHC, respectively. CD45 positive cells in the cancer cell compartment were counted in tissue sections where the partition between side A and side B was visually apparent. Images of the CD45 cells and cancer cells (or their nuclei) were converted into binary “masks” using ImageJ, for easy of visualization and are shown in FIG. 11.

To quantify anti-cancer effects of the immune cells, killing of the cancer cells was monitored by IHC of the cleaved caspase-3 apoptosis marker. Apoptotic cells are presented as a percentage of the total cancer cells in the imaged field.

### Results

TABLE 4
Immune cell types and their infiltration score, and cancer cell killing score.

| | | # tumors | | | |
|---|---|---|---|---|---|
| | | showing | | | |
| | | immune cell | # immune | | |
| | | infiltration | cell- | | |
| | # tumors | of the total | infiltrated | Infiltration | Cancer cell |
| | showing | tumors | tumors | score (# | killing (% |
| | partition of | showing | showing | CD45 cells/ | caspase 3 |
| Immune | side A and side | partition | elevated | area of side | positive |
| cell type | B (total, n) | [X (Y)] | apoptosis | A) | cells) |
| TILs #2 | 7 (10) | 5 (7) | 0 | 100 ± 30 | <1 |
| TILs #3 | 7 (11) | 6 (7) | 5 (6) | 64 ± 13 | 9 ± 4 |
| TILs #4 | 6 (8) | 0 (8) | 0 | 0 | 1 |
| PBMCs | 7 (10) | 7 | 0 | 130 ± 35 | 1 |
| CAR-T | 1 (1) | 1 | 1 | 22* | 32 |

*CAR-T destroyed the cancer cells and tumor structure in 9/10 of the tumors. Only one tumor showed partition and remaining cancer cells.

Four days (96 h) after engraftment tumors from two CAR-T cell types were mostly devoid of cancer cells, with T cells dispersed throughout the tumor. There were only a few tumors that showed residual cancer cell masses and a discernable partition of side A and side B (shown in FIGS. 11Q-11T).

The infiltration propensity of the tested cell types was CAR-T >>PBMC >TILs #2>TILs #3, while TILs #4 did not infiltrate at all. The level of killing activity was CAR-T >>>TILs #3, while TILs #2, TILs #4 and PBMC showed little if any cancer cell killing.

### Example 7

### Infiltrating Properties of Non-Immune Cells into a Cancer Cell Tumor Mass

The inventors examined infiltration properties of non-immune cells (suspended within side B) into a cancer cell tumor mass (suspended within side A) whereby conditions of side A of the fusion tumor are suited to the non-immune cells, while the conditions of side B are suited to the growth of the cancer cells.

### Method

HUVEC (Human Umbilical Vascular Endothelial Cells; Lonza) primary low passage cells were grown in EGM2 medium (Lonza), as per manufacturer instructions. Varying numbers of chilled HUVEC were resuspended in cold Matrigel (15 μl per pellet) containing basic fibroblast growth factor (bFGF; Peprotec) and solidified on a Parafilm surface and placed in a humidified incubator at 37° C. for 30 minutes to allow solidification; similarly, A2780 ovarian carcinoma cells were resuspended in Matrigel® (15 μl per pellet) containing 30 ng/ml bFGF and solidified on a Parafilm surface at 37° C.

Matrix pellet pairs (one “side A” and one “side B”) were placed adjacently (˜1 mm gap between matrix pellet rims) on the CAM of E9 eggs, sealed and returned to the egg incubator.

At E17 (8 days after engraftment), tumors were harvested by excision from the egg, rinsed in PBS, fixed in 4% (w/v) paraformaldehyde for 30 minutes, rinsed in PBS and transferred to 70% (v/v) ethanol prior to embedding in paraffin by standard techniques.

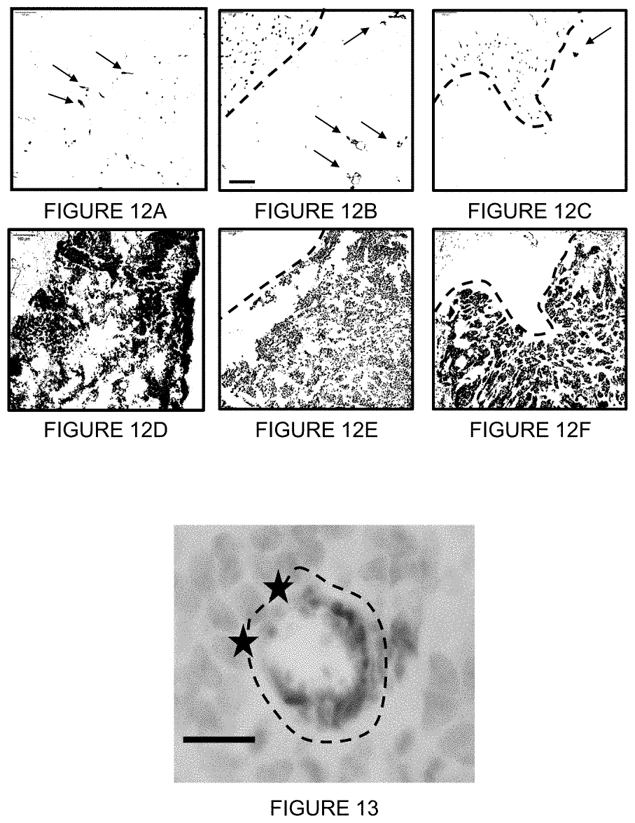

Tumors embedded in paraffin were sectioned in a microtome (6 μm thickness) and stained by immunohistochemistry for markers. CD31 was used to detect human vasculature endothelial cells, A2780 cells were identified using CD155. CD31 positive vascular structures (elongated tubules containing chick blood cells) in the cancer cell compartment were counted in tissue sections where the border between side A and side B was visually apparent. The area of side A was calculated using ImageJ software and a score was given as the number of CD31 positive vessels/area (mm²)—right hand column in Table 3. Images of the CD31 cells and CD155 cells were converted into binary for ease of visualization and are shown in FIG. 12.

### Results

TABLE 5
Cell types and their infiltration score.

| | | | | # CD31 | # CD31 |
|---|---|---|---|---|---|
| | | | | vessels - Side | vessels -Side |
| Group # | | Side A | Side B | B/tumor | B/area (mm²) |
| 1 | cell type | HUVEC | A2780 | 3.83 ± 2.33 | 8.95 ± 4.9 |
| | | (stem-cell | (ovarian | (n = 6) | (n = 6) |
| | | like | cancer) | **0.184 | **0.098 |
| | | endothelial) | | | |
| | Matrix | Matrigel ®, | Matrigel ®, | | |
| | conditions | 10 ng bFGF | 30 ng bFGF | | |
| 2 | cell type | HUVEC | A2780 | 12.25 ± 6.33 | 31.83 ± 13.4 |
| | | (stem-cell | (ovarian | (n = 4) | (n = 4) |
| | | like | cancer) | | |
| | | endothelial) | | | |
| | Matrix | Matrigel ®, | Matrigel ®, | | |
| | conditions | 1 ng bFGF | 30 ng bFGF | | |
| 3 | cell type | HUVEC | A2780 | 0.5 ± 0.5 | 1.38 ± 1.4 |
| | | (stem-cell | (ovarian | (n = 4) | (n = 4) |
| | | like | cancer) | **0.1137 | **0.065 |
| | | endothelial) | | | |
| | Matrix | Matrigel ®, | Gelatin | | |
| | conditions | 10 ng bFGF | sponge, | | |
| | | | 30 ng bFGF | | |
| 4 | cell type | HUVEC | A2780 | 4.5 ± 2.13 | 15.97 ± 8.8 |
| | | (stem-cell | (ovarian | (n = 6) | (n = 6) |
| | | like | cancer) | **0.208 | **0.330 |
| | | endothelial) | | | |
| | Matrix | Matrigel ® | Matrigel ® | | |
| | conditions | only | only | | |
| 5 | cell type | HUVEC | Mel-624 | 6 ± 3.46 | 18.14 ± 9.8 |
| | | (stem-cell | (melanoma) | (n = 3) | (n = 3) |
| | | like | | **0.6131 | **0.479 |
| | | endothelial) | | | |
| | Matrix | Matrigel ®, | Matrigel ®, | | |
| | conditions | 10 ng/ml bFGF | 30 ng/ml bFGF | | |

HUVEC engrafted on side A stained for CD31 showed small clusters or individual cells dispersed throughout the pellet, while CD31 staining on side B revealed that there are tubular structures containing chick blood cells, indicating that the HUVEC have infiltrated from side A into side B and differentiated into mature blood vessels. The inventors observed that the blood vessels containing CD31 positive cells often comprise CD31 negative cells, indicating that these are chick vascular endothelial cells that have organized into the structure of a chimeric blood vessel. In FIG. 13, a tissue section from a HUVEC—A2780 fusion tumor has been stained with anti-CD31 (DAB) and hematoxylin (all nuclei), and in this image is a circular blood vessel has been outlined with a dashed line, comprising mostly of DAB stained cells, however there are a few cells that do not express CD31 (indicated by a star symbol) that are incorporated in the blood vessel, indicating that these blood vessels comprise both human and chick endothelial cells.

When the HUVEC and A2780 cells were mixed together in a single Matrigel® pellet, both morphological forms of CD31 positive cells were observed: small dense clusters, and elongated tubules, indicating that only a fraction of the HUVEC cells are incorporated into growing chick blood vessels. Thus, engrafting HUVEC and A28780 in separate adjacent matrix pellets, provides the conditions such that all CD31 positive cells in the A2780 compartment are indeed incorporated into blood vessels.

When melanoma cell line Mel-624 was placed on side A, little HUVEC infiltration was observed, possibly due to lower levels of pro-angiogenic factors being secreted by the cancer cells, compared to A2780 cells. Similarly, when side B matrix scaffold material was changed from Matrigel® to gelatin sponge (Spongostan®), CD31 positive blood vessels infiltrating side B were not observed.

In the absence of bFGF growth factor from the Matrigel® pellet, human blood vessel formation was reduced by half (Table 3, Group 4).

### Example 8

### Retinal Pigment Epithelial (RPE) Cells and Age-Related Macular Degeneration

Eye disease such as wet age-related macular degeneration (AMD) is thought to be caused by neo-angiogenesis in the retina brought about by oxygen deprivation in the retina that induces the retinal pigment epithelia cells to secrete pro-angiogenic signals. Aberrant vasculature formation in the retina causes blood vessel leakiness which results in blurred vision.

RPE cells can be grown in cell culture and engrafted on the CAM in a suitable hydrogel that supports their differentiation into RPE. The hydrogel may comprise or consist of collagen IV, laminin, fibronectin, or combination thereof, all of which are extracellular membrane proteins, which are known to support RPE attachment and function.

Human endothelial cells are grown in one hydrogel (e.g., Matrigel), and RPE cells in a second hydrogel comprising collagen IV, laminin, and fibronectin, and engrafted adjacently (e.g., 1-5 mm apart) on the CAM. Subsequent vascularization of the RPE cell mass by human endothelial cells is observed (e.g., by microscopy, including fluorescent microscopy and immunohistochemistry) and quantified. Induction of hypoxia in the RPE cell mass that may occur during the engraftment process is observed using a metabolic marker, such as Hypoxyprobe®, or the like, or hypoxia may be brought about directly by various pretreatments of the RPE cells prior to engraftment, or by adding hypoxia inducing agents to the hydrogel prior to engraftment. Alternatively, engraftment of RPE cells in collagen I hydrogel may be sufficient to induce a proangiogenic signal in the RPE side of the growth, that induces the human endothelial cell infiltration of the RPE mass. Therefore, the described system is suitable for detecting dynamics of RPE vascularization, and accordingly enables screening for compounds capable of inhibiting or reducing such vascularization.

### Example 9

### Hepatocytes and Fatty Liver

Liver disease such as fatty liver occurs predominantly in aging livers, and has been linked to impaired vasculature function. In this type of disease, angiogenesis increases, but contributes to inflammation and fibrosis, while impairing normal liver regeneration.

Hepatocytes and liver sinusoidal endothelial cells are dissociated from either young or aging mice (or humans) and engrafted in separate hydrogels to examine the interaction between the two cells types. Young healthy hepatocytes are matched with aged endothelial cells from diseased fatty liver, or young endothelial cells are matched with aged fatty liver hepatocytes, in order to study and model fatty liver disease.

The hydrogels that best support the two cell types are determined initially, and then the two cell types are co-engrafted on the same CAM, as described above. Therefore, the described system is suitable for detecting dynamics of hepatocytes and endothelial cells, and particularly enables screening for compounds capable of redressing the balance between endothelial cell and hepatocyte inflammatory signaling.

### Example 10

### Adipose Tissue Vasculature and Fat Tissue Inflammation

Obesity and related disorders have been linked to endothelial dysfunction, through excess angiogenesis and/or inflammation. Adipose tissue is a source of signaling to both the vasculature and the immune system, thus models that incorporate human vasculature and adipocytes for quantifying inflammation and other cross-talk events may be useful for studying therapeutics and therapeutic strategies for inhibiting the inflammatory reaction in adipose tissue.

Adipose cells engrafted in a suitable matrix scaffold, with human endothelial cells, either from healthy donors or from obese donors (expressing inflammatory markers) are engrafted in adjacently (e.g., 1-5 mm apart) placed matrix pellets. Effects of inflamed vasculature on adipocytes are quantified where the human endothelial cells have been incorporated into chick blood vessels within the adipocyte cell mass.

Therefore, the described system is suitable for studying adipose tissue inflammation and/or subsequent obesity development, and accordingly enables screening for compounds capable of inhibiting or reducing fat tissue inflammation, obesity, a metabolic syndrome symptom, or any combination thereof.

### Example 11

### Hematopoietic Stem Cell Bone Marrow Niche and Bone Regeneration

Bone regeneration and remodeling in health and disease can be modeled for the assaying of therapies using complex tissue models comprising multiple cell types, from vasculature cells, fibroblasts (bone marrow mesenchymal stem cells), hematopoietic cells, and bone marrow cells (osteoblasts, osteoclasts etc.). The interaction of these multiple cell types can be monitored by growing the cells together in a suitable matrix support, allowing quantification of the effects of manipulations.

In vitro methods have been described, but there are no suitable in vivo models that allow for the precise building of complex tissues mimicking the human bone marrow-vasculature interaction. Using the herein disclosed CAM system, scaffolds mimicking bone structures (e.g., hydrogels, bone pieces or other types of scaffold material) are mixed with the different cell types and engrafted on the CAM. Vasculature cells are engrafted in an adjacent (e.g., 1-5 mm apart) matrix pellet, allowing for vascularization of the bone (or bone mimic) and hematopoietic niches by chimeric chick—human blood vessels. Arrival and departure of human hematopoietic cells or fibroblasts are monitored in real time by live imaging of fluorescently labeled cells.

Therefore, the described system is suitable for studying bone remodeling and regeneration, and accordingly enables screening for compounds capable of inducing, promoting, enhancing, or any combination thereof, of such bone morphogenic process.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A mammalian-avian chimeric model system comprising:
a. a fertilized avian egg comprising a chorioallantoic membrane (CAM);
b. a first type of a mammalian cell; and
c. a second type of a mammalian cell,
wherein said first type of a mammalian cell and said second type of a mammalian cell are dispersed in separate hydrogels, and wherein said separate hydrogels are at a distance of not more than 5 mm from one another on said CAM.

2. The system of claim 1, wherein anyone of said separate hydrogels comprises 50,000 to 1,000,000 cells.

3. The system of claim 1, wherein said first type of a mammalian cell is a proliferating cell or a differentiating cell.

4. The system of claim 3, wherein said proliferating cell is an abnormally proliferating cell or a cancerous cell.

5. The system of claim 1, wherein said second type of a mammalian cell is an immune cell, an endothelial cell, or any progenitor cells thereof.

6. The system of claim 5, wherein said immune cell is selected from the group consisting of: an infiltrating cell, a cytokine-mediated remote killing cell, and a cell-cycle arresting cell.

7. The system of claim 5, wherein said immune cell is a lymphocyte or a myeloid cell.

8. The system of claim 1, further comprising a therapeutic agent selected from the group consisting of: a chemical, a molecule, a polypeptide, a cell, and a virus.

9. The system of claim 1, further comprising one or more stimulatory agents, wherein said one or more stimulatory agents increases one or more cellular activities selected from the group consisting of: proliferation, growth, survival, motility, angiogenesis, neo-vascularization, and cytotoxicity.

10. The system of claim 4, wherein at least a portion of said cancerous cells are in a form of a tumor or a cystoid.

11. A method for preparing a mammalian-avian chimeric model system, comprising:
a. providing a fertilized avian egg comprising a CAM;
b. grafting a first type of a mammalian cell dispersed in a hydrogel to said CAM; and
c. grafting a second type of a mammalian cell dispersed in a hydrogel to said CAM,
thereby preparing a mammalian-avian chimeric model system.

12. The method of claim 11, wherein each of said hydrogels is provided to said CAM at a distance of not more than 5 mm from one another.

13. The method of claim 11, wherein said hydrogels are grafted to said CAM on embryonic day 6 (E6) to embryonic day 17 (E17).

14. (canceled)

15. (canceled)

16. (canceled)

17. (canceled)

18. The method of claim 11, further comprising a step of determining the engraftment of said first type of a mammalian cell to said CAM.

19. The method of claim 11, further comprising providing one or more stimulatory agents to one or more of said first type of a mammalian cell and said second type of a mammalian cell, wherein said one or more stimulatory agents increases one or more cellular activities selected from the group consisting of: proliferation, growth, survival, motility, angiogenesis, neo-vascularization, and cytotoxicity.

20. (canceled)

21. (canceled)

22. The method of claim 19, wherein said providing is by: contacting one or more of said first type of a mammalian cell and said second type of a mammalian cell, an intravenous injection into the CAM blood vessels, or a combination thereof.

23. The method of claim 11, further comprising a step of determining the effector activity of said second type of a mammalian cell, optionally wherein said effector activity is selected from the group consisting of: infiltration, cytokine secretion, cytokine-mediated remote killing, and cell-cycle arresting.

24. (canceled)

25. A method for determining an interaction between a first type of a mammalian cell and a second type of a mammalian cell, comprising:
a. providing the mammalian-avian chimeric model system of claim 1; and
b. determining at least one of: (a) cellular phenotype in said first type of a mammalian cell; and (b) an effector activity in said second type of a mammalian cell,
wherein a change of said phenotype in said first type of a mammalian cell and/or a change of said effector activity in said second type of a mammalian cell compared to control, is indicative of an interaction between a first type of a mammalian cell and a second type of a mammalian cell.

26. The method of claim 25, further comprising a step of contacting said mammalian-avian chimeric model system with one or more stimulatory agent, wherein said one or more stimulatory agents increases one or more activities selected from the group consisting of: proliferation, growth, survival, motility, angiogenesis, neo-vascularization, and cytotoxicity.

27. The method of claim 25, wherein (i) said cellular phenotype of said first type of a mammalian cell is selected from the group consisting of proliferation rate, cell death rate, differentiation, tumorigenesis, and metabolic rate (ii) said effector activity of said second type of a mammalian cell is selected from the group consisting of: infiltration, cytokine secretion, cytokine-mediated remote killing, cell-cycle arresting, angiogenesis, and neo-vascularization; or combination of (i) and (ii).

28.-30. (canceled)
